(19) United States
(12) Patent Application Publication
(10) Pub. No.: US 2021/0068750 A1
(43) Pub. Date: Mar. 11, 2021

(54) Three-Dimensional Biopsy Imaging and Planning System and Method

(71) Applicant: 3DBiopsy, Inc., Aurora, CO (US)

(72) Inventor: Nelson N. Stone, Vail, CO (US)

(21) Appl. No.: 16/565,176

(22) Filed: Sep. 9, 2019

(51) Int. Cl. A61B 5/00; A61B 10/02; A61B 90/00; G06T 17/00

(52) U.S. Cl. CPC A61B 5/4887; A61B 10/0233; A61B 90/37; G06T 2210/41; G06T 17/00; A61B 2010/0208; A61B 5/7425

(57) ABSTRACT

A system and method are disclosed for three-dimensional biopsy imaging and planning. Elements include a computer coupled with a database, imaging system, actuator assembly, and pathology collection device, such as a needle assembly connected to the actuator assembly. The computer is configured to operate the imaging system to acquire one or more tissue images from an animal. The computer is further configured to generate a three-dimensional tissue model from the tissue image(s) and to develop a biopsy plan to collect tissue specimens from one or more tissue specimen collection sites on the three-dimensional tissue model. The computer actuates the actuator assembly to extend the needle assembly into the animal to collect one or more tissue specimens, displays biopsy results associated with each of the one or more collected tissue specimens on the three-dimensional tissue model, and generates a treatment plan to treat one or more tissue regions of the animal.

### TECHNICAL FIELD

The present disclosure relates generally to medical imaging and biopsy planning technology and specifically to a system and method of planning and conducting biopsies, imaging pathology results, and developing treatment plans.

### BACKGROUND

Planning and executing a biopsy may involve capturing a plurality of tissue images using medical imaging technology, generating a three-dimensional (3D) model of the organ or tissue to be biopsied, and then creating a biopsy plan selecting individual tissue specimen collection sites for biopsy on the 3D model. If a medical professional determines that changes in biopsy plan tissue specimen collection sites are necessary to adequately biopsy an organ or tissue, the biopsy plan must be re-generated and checked before biopsy can occur. The inability to modify biopsy plans with the biopsy in progress, and to visualize the effects the modifications will have on tissue specimen collection sites, tissue volumes collected, and potentially unsampled regions of tissue, is undesirable.

### BRIEF DESCRIPTION OF THE DRAWINGS

A more complete understanding of the present invention may be derived by referring to the detailed description when considered in connection with the following illustrative figures. In the figures, like reference numbers refer to like elements or acts throughout the figures.

FIG. 1 illustrates an exemplary system, according to a first embodiment;

FIG. 2 illustrates the 3D mapping system of FIG. 1 in greater detail, according to an embodiment;

FIG. 3 illustrates an exemplary method of imaging, planning, and conducting a tissue biopsy, developing a treatment plan, and implementing a treatment plan, according to an embodiment;

FIG. 4 illustrates an exemplary method of conducting a biopsy planning phase, according to an embodiment;

FIGS. 5A-5B illustrate exemplary contouring visualization interfaces, according to embodiments;

FIGS. 6A-6B illustrate exemplary biopsy planning interfaces, according to embodiments;

FIGS. 7A-7B illustrate exemplary biopsy visualization interfaces, according to embodiments;

FIG. 8 illustrates a biopsy planning module displaying a tissue gap on a biopsy visualization interface, according to an embodiment;

FIG. 9 illustrates an exemplary method of conducting a biopsy phase, according to an embodiment;

FIG. 10 illustrates an exemplary tissue specimen collection visualization interface, according to an embodiment;

FIG. 11 illustrates an exemplary method of conducting a pathology phase, according to an embodiment;

FIGS. 12A-12B illustrate exemplary pathology visualization interfaces, according to embodiments; and

FIG. 13 illustrates an exemplary method of conducting a treatment phase, according to an embodiment.

### DETAILED DESCRIPTION

Aspects and applications of the invention presented herein are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

As described more fully below, aspects of the following disclosure relate to a three-dimensional (3D) tissue biopsy imaging and planning system and method. Embodiments of the following disclosure provide a pathology collection device, such as a needle assembly connected to an actuator assembly, configured to collect tissue specimens from a human patient or animal, an imaging system, and a 3D mapping system operatively associated with the needle assembly, actuator assembly, and imaging system. According to a first aspect, embodiments display tissue images, generate a 3D tissue model based on one or more tissue images, and prepare a biopsy plan to collect one or more tissue specimens from selected tissue locations using the needle assembly and actuator assembly. According to a second aspect, embodiments collect one or more tissue specimens for biopsy, and record and display on the 3D tissue model the location from which the needle assembly removed each tissue specimen. According to a third aspect, embodiments display the location of lesions located via tissue specimen biopsy on the 3D tissue model and visualize the effect of possible treatment procedures.

Embodiments of the following disclosure streamline the process of collecting multiple tissue specimens with a high degree of spatial precision and reduce the risk of specimen misattribution and/or numbering errors. Embodiments generate flexible biopsy plans that automatically adapt to the addition or subtraction of tissue specimen collection sites during the planning or biopsy phase. Embodiments highlight, on the 3D tissue model, areas of the tissue that the current biopsy plan does not sample and which may harbor lesions, and continually update this information as the biopsy is planned and executed. In addition, or as an alternative, embodiments visualize the real-time location of one or more needle assemblies inserted into human or animal tissue, and adjust the biopsy plan to reflect the actual location in 3D space in which the needle assembly was inserted.

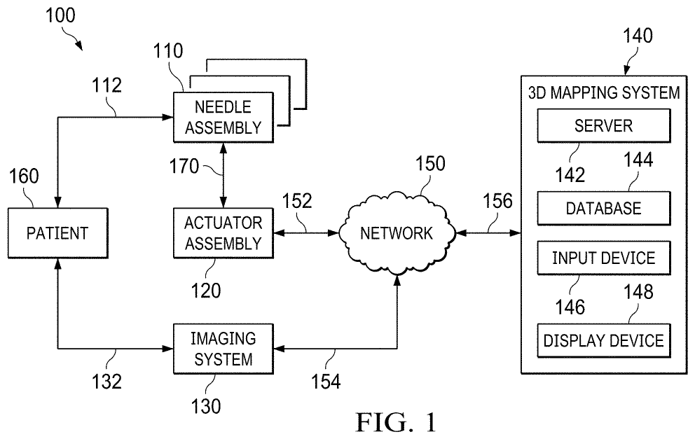

FIG. 1 illustrates exemplary system 100 according to a first embodiment.

System 100 comprises one or more needle assemblies 110, actuator assembly 120, imaging system 130, 3D mapping system 140, network 150, and patient 160. Although a plurality of needle assemblies 110 and a single actuator assembly 120, imaging system 130, 3D mapping system 140, network 150, and patient 160 are shown and described, embodiments contemplate any number of needle assemblies 110, actuator assemblies 120, imaging systems 130, 3D mapping systems 140, networks 150, or patients 160, according to particular needs.

In one embodiment, needle assembly 110 operates in conjunction with actuator assembly 120 to remove a tissue specimen from a target tissue and to store the tissue specimen until pathological examination. Target tissue may be any animal tissue type or organ, including but not limited to human patient target tissue. Tissue types include, but are not limited to, epithelial tissue, connective tissue, muscle tissue, and/or nervous tissue. Organs include, but are not limited to, prostate, breast, kidney, and liver.

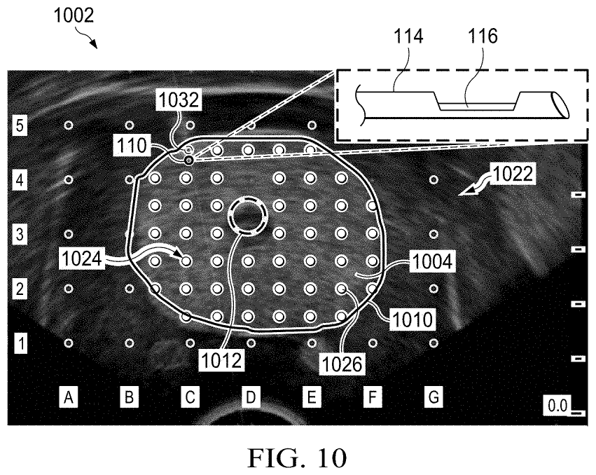

Needle assembly 110 comprises inner mandrel 114 that travels within an outer cannula. As best illustrated by FIG. 10, mandrel 114 includes an elongated cylindrical body, extending from a proximal cylindrical body end to a distal cylindrical body end. The cylindrical body has an outer diameter. In an embodiment, the cylindrical body outer diameter is from about 0.1842 mm to about 4.572 mm, preferably from to about 1.27 mm to about 2.77 mm. The cylindrical body is manufactured from a surgically suitable material, including stainless steel. A generally rectangular first connecting element forming an offset aperture is located at the proximal end of the cylindrical body for connecting mandrel 114 to actuator assembly 120. The first connecting element is molded to the proximal end of the cylindrical body forming a collar about the cylindrical body. The distal end of the cylindrical body forms a tip that terminates at a point. The cylindrical body forms adjacent core bed 116 extending between first core bed 116 end, and second core bed 116 end located adjacent the tip, providing a cavity for retaining a tissue specimen. In some embodiments, the second end of core bed 116 is about 5 mm from the terminating point of the cylindrical body. Core bed 116 presents inner core bed 116 surface bound laterally by first core bed 116 longitudinal edge and an opposite second core bed 116 longitudinal edge, and extending from a distal end by second core bed 116 end, and from a proximal end by first core bed 116 end.

Needle assembly 110 core bed 116 has a longitudinal length between the first core bed 116 end and second core bed 116 end from about 1 mm to about 200 mm, preferably from about 20 mm to about 60 mm. In an embodiment, core bed 116 forms an upwardly open lower core bed 116 cavity whereby a substantial portion of inner core bed 116 surface lies below central core bed 116 axis between adjacent first and second core bed 116 longitudinal edges. Core bed 116 longitudinal edges are at or above a horizontal plane coincident with the central axis thereby improving the stiffness of the cylindrical body at core bed 116, and thus the cross sectional moment of inertia. In an embodiment, lower core bed 116 cavity is formed from core bed 116 having a C-shaped cross sectional configuration presenting a trough-shaped cavity with the bottom of the cavity disposed below central core bed 116 axis. In other embodiments, and as best illustrated by FIG. 10, core bed 116 comprises a flat bottom surface bordered by perpendicular walls, such that core bed 116 presents a rectangular cross sectional configuration with the bottom of the cavity disposed below central core bed 116 axis.

According to embodiments, needle assembly 110 core bed 116 forms one or more core bed 116 projections with a contact surface for one or more of marking, securing, impressing, engaging, orientating, and scoring a tissue specimen within core bed 116. In some embodiments core bed 116 projection includes a plurality of adjacent longitudinal ridges formed by inner core bed 116 surface extending the length of core bed 116 between first core bed 116 end and second core bed 116 end that contact the tissue specimen. In some embodiments core bed 116 projection includes a plurality of adjacent transverse ridges formed by inner core bed 116 surface extending between first core bed 116 longitudinal edge and second core bed 116 longitudinal edge spaced between first core bed 116 end and the second core bed 116 end that contact the tissue specimen.

Actuator assembly 120 extends and retracts one or more needle assemblies 110 to excise one or more tissue specimens from a target tissue site. In an embodiment, actuator assembly 120 includes an actuator body extending from a proximal actuator end to a distal actuator end forming actuator sidewalls, including a front actuator wall at the distal actuator end, and a rear actuator wall at the proximal actuator end. An opening in the front actuator wall allows needle assembly 110 to extend from actuator assembly 120. A cover is movable on the actuator body allowing access to the interior of actuator assembly 120.

Actuator assembly 120 includes an actuator for axially moving needle assembly 110 mandrel 114 and an actuator for axially moving needle assembly 110 cannula. Mandrel 114 actuator member includes mandrel 114 guide extending from a proximal actuator end at the rear actuator wall, to a distal actuator end at the front actuator wall. Mandrel 114 carriage assembly moves axially along mandrel 114 guide for moving mandrel 114 within the cannula. A biasing member, such as mandrel 114 spring, is disposed between mandrel 114 carriage assembly and the rear actuator wall, and when compressed, biases mandrel 114 carriage assembly toward the distal actuator end of actuator assembly 120. Mandrel 114 pin extending from mandrel 114 carriage assembly receives the first connecting element of mandrel 114. A notch in mandrel 114 carriage assembly receives a first firing pin when mandrel 114 carriage assembly is moved to the distal actuator end. A first carriage stop disposed between mandrel 114 carriage assembly and the front actuator wall limits the forward movement of mandrel 114 carriage assembly along mandrel 114 guide.

A cannula actuator member for moving needle assembly 110 cannula is disposed adjacent mandrel 114 actuator member. The cannula actuator member includes a cannula guide extending from a proximal actuator end at the rear actuator wall, to a distal actuator end at the front actuator wall. A cannula carriage assembly moves axially along the cannula guide for moving the cannula about mandrel 114. A biasing member, such as a cannula spring, is disposed between the cannula carriage assembly and the rear actuator wall, and when compressed, biases the cannula carriage assembly toward the distal actuator end of actuator assembly 120. A cannula pin extending from the cannula carriage assembly receives the second connecting element of the cannula. A notch in the cannula carriage assembly receives a second firing pin when the cannula carriage assembly is moved to the distal actuator end. A second carriage stop disposed between the cannula carriage assembly and the front actuator wall limits the forward movement of the cannula carriage assembly along the cannula guide.

An adjusting member allows an operator to move the first carriage stop and second carriage stop. The adjusting member includes an externally threaded member disposed between mandrel 114 guide and cannula guide, and extend between a proximal end rotatably disposed adjacent the rear actuator wall, and a distal end rotatably disposed at the front actuator wall. A wheel at the exterior of the actuator body is operably connected to the threaded member for rotating the threaded member. A threaded surface on the first carriage stop, and a threaded surface on the second carriage stop interface with the threaded member whereby rotation of the threaded member moves the threaded members along their respective guides. According to embodiments, the distance between the distal edge of mandrel 114 carriage assembly and proximal edge of the first carriage stop can be adjusted from about 0 mm to about 70 mm; in other embodiments, from about 20 mm to about 60 mm. An indicator on the wheel, or at another location on actuator assembly 120, provides an indication of the distance between mandrel 114 carriage assembly and first carriage stop, and thus, the distance the cannula will travel through a target tissue during the collection of a tissue specimen.

In an embodiment, imaging system 130 comprises an imaging device capable of creating visual representations of the interior tissues of a human or animal. Imaging system 130 generates transverse and sagittal images of target tissue and transmits target tissue images to 3D mapping system 140. According to embodiments, imaging system 130 may comprise one or more probes that contact the skin or the internal structure or organ of a human or animal and provide images of internal tissue. In an embodiment, imaging system 130 may comprise an ultrasound imaging system that uses, for example, ultrasonic frequencies of 2 megahertz or higher to image internal tissue structures. In other embodiments, imaging system 130 may comprise, for example, a magnetic resonance imaging (MRI) system or other scanning device.

3D mapping system 140 may comprise a system operatively associated with needle assembly 110, actuator assembly 120, and imaging system 130. 3D mapping system 140 may be configured to generate one or more 2D or 3D tissue models based on one or more tissue images captured by imaging system 130, to prepare a biopsy site plan to collect one or more tissue specimens using needle assembly 110 and actuator assembly 120, and to display on the 3D tissue model the locations of lesions located via tissue specimen biopsy. Although 3D mapping system 140 is primarily shown and described as generating 3D tissue models based on one or more tissue images, embodiments contemplate 3D mapping system 140 generating any number or configuration of 2D or 3D tissue models based on one or more tissue images, according to particular needs.

In an embodiment, 3D mapping system 140 may comprise server 142 and database 144. Server 142 may be programmed to control needle assembly 110, actuator assembly 120, and imaging system 130, and to generate a 3D tissue model based on one or more tissue images. Database 144 may comprise one or more databases or other data storage arrangements at one or more locations local to, or remote from, 3D mapping system 140. In one embodiment, one or more databases 144 is coupled with one or more servers 142 using one or more local area networks (LANs), metropolitan area networks (MANs), wide area networks (WANs), network 150, the Internet, or any other appropriate wire line, wireless link, or any other communication link. One or more databases 144 stores data that is made available and may be used by one or more servers 142 according to the operation of system 100. In addition, and as discussed herein, 3D mapping system 140 may comprise a cloud-based imaging and processing system comprising processing and storage devices at one or more locations, local to, or remote from system 100 and one or more needle assemblies 110, actuator assemblies 120, and/or imaging systems 130.

Server 142 may include one or more processors and associated memory to execute instructions and manipulate information according to the operation of system 100 and any of the methods described herein. One or more server 142 processors may execute an operating system program stored in memory to control the overall operation of server 142, 3D mapping system 140, and/or one or more needle assemblies 110, actuator assemblies 120, or imaging systems 130. For example, one or more server 142 processors may control the reception and transmission of signals within system 100. One or more server 142 processors may execute other processes and programs resident in memory, such as, for example, registration, identification or communication, and may move data into or out of the memory, as required by an executing process.

In addition, or as an alternative, 3D mapping system 140 may comprise input device 146 and display device 148. Input device 146 may include any suitable input device, such as a keypad, mouse, touch screen, microphone, or other device to input information into a computer. Display device 148 may comprise any suitable device that may convey information associated with the operation of communication platform 100, including digital or analog data, visual information, or audio information. In an embodiment, display device 148 may comprise, for example, a CRT monitor having a resolution of 720×480 pixels or greater, and may be connected to server 142 by an S-video cable or other type of cable. In other embodiments, display device 148 may comprise, for example, an LCD monitor having a resolution of 1,920×1,080 pixels or greater, and may be connected to server 142 by an HDMI cable.

In an embodiment, network 150 includes the Internet and any appropriate local area networks (LANs), metropolitan area networks (MANs), or wide area networks (WANs) coupling actuator assembly 120, imaging system 130, and 3D mapping system 140. For example, data may be maintained local to, or externally of, actuator assembly 120, imaging system 130, and 3D mapping system 140, and made available to one or more of actuator assembly 120, imaging system 130, and 3D mapping system 140 using network 150 or in any other appropriate manner. For example, data may be maintained in a cloud database at one or more locations external to actuator assembly 120, imaging system 130, and 3D mapping system 140, and made available to one or more of actuator assembly 120, imaging system 130, and 3D mapping system 140 using cloud architecture or in any other appropriate manner.

In one embodiment, actuator assembly 120 may be coupled with network 150 using communication link 152, which may be any wireline, wireless, or other link suitable to support data communications between actuator assembly 120 and network 150 during operation of system 100. Imaging system 130 may be coupled with network 150 using communication link 154, which may be any wireline, wireless, or other link suitable to support data communication between imaging system 130 and network 150 during operation of system 100. 3D mapping system 140 may be coupled with network 150 using communication link 156, which may be any wireline, wireless, or other link suitable to support data communications between 3D mapping system 140 and network 150 during operation of system 100. Although communication links 152-156 are shown as generally coupling actuator assembly 120, imaging system 130, and 3D mapping system 140 to network 150, any of actuator assembly 120, imaging system 130, and 3D mapping system 140 may communicate directly with each other using wired cables, fiber optic lines, or any other direct wired or wireless communication connections, according to particular needs.

In an embodiment, patient 160 comprises the human or animal on which system 100 performs the actions described in greater detail below. Patient 160 may comprise any human or animal. 3D mapping system 140 may image any portion or organ of any human or animal. In an embodiment, imaging system 130 may image the tissue of patient 160 using contact 132, which may comprise, for example, one or more imaging probes that contact the skin of patient 160 and provide images of internal tissue. In other embodiments, contact 132 may comprise any non-contact or wireless interaction between patient 160 and imaging system 130, such as, for example, a magnetic resonance imaging scan. In an embodiment, needle assembly 110 may contact patient 160 using contact 112, which may comprise, for example, one or more needles, cannulas, and/or mandrel 114s injections into the tissue of patient 160.

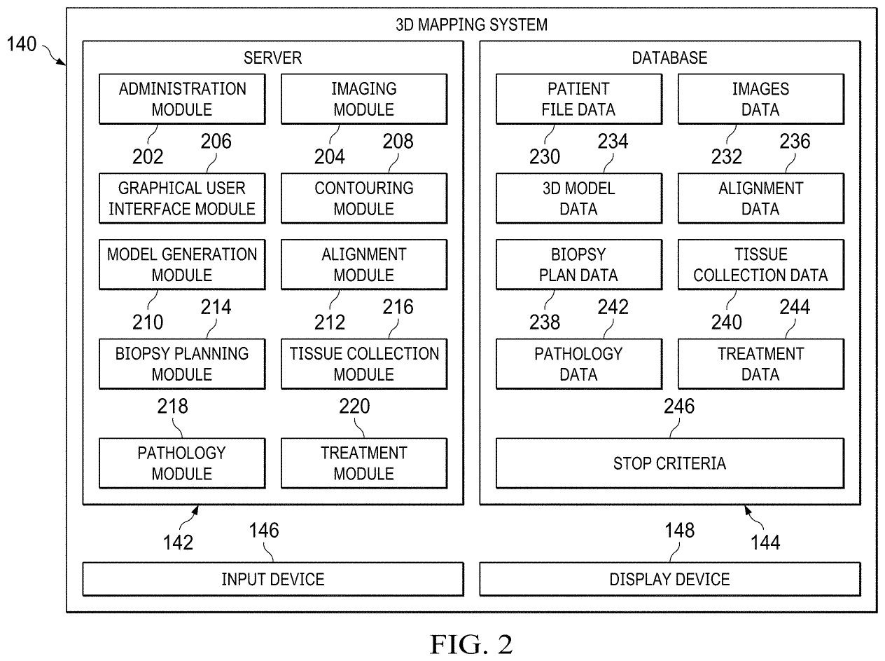

FIG. 2 illustrates 3D mapping system 140 of FIG. 1 in greater detail, according to an embodiment. As described above, 3D mapping system 140 comprises sever 142, database 144, input device 146, and display device 148. Although 3D mapping system 140 is shown and described as comprising a single server 142, database 144, input device 146, and display device 148, embodiments contemplate any suitable number or combination of servers 142, databases 144, input devices 146, and/or display devices 148 internal to or externally coupled with 3D mapping system 140.

Server 142 of 3D mapping system 140 may comprise administration module 202, imaging module 204, graphical user interface module 206, contouring module 208, model generation module 210, alignment module 212, biopsy planning module 214, tissue collection module 216, pathology module 218, and treatment module 220. Although server 142 is shown and described as comprising an administration module 202, imaging module 204, graphical user interface module 206, contouring module 208, model generation module 210, alignment module 212, biopsy planning module 214, tissue collection module 216, pathology module 218, and treatment module 220, embodiments contemplate any suitable number or combination of modules located at one or more locations, local to, or remote from 3D mapping system 140, such as on multiple servers or computers at any location in system 100.

Database 144 of 3D mapping system 140 may comprise one or more databases or other data storage arrangements at one or more locations, local to, or remote from, server 142. Database 144 comprises, for example, patient file data 230, images data 232, 3D model data 234, alignment data 236, biopsy plan data 238, tissue collection data 240, pathology data 242, treatment data 244, and stop criteria 246. Although database 144 is shown and described as comprising patient file data 230, images data 232, 3D model data 234, alignment data 236, biopsy plan data 238, tissue collection data 240, pathology data 242, treatment data 244, and stop criteria 246, embodiments contemplate any suitable number or combination of these, located at one or more locations, local to, or remote from, 3D mapping system 140, according to particular needs.

Administration module 202 of server 142 may configure, update, and/or manage the operation of 3D mapping system 140 and system 100. That is, administration module 202 may configure, update, and/or manage the broader operation of system 100 and change which instructions and data are executed by and/or stored in 3D mapping system 140 or in needle assembly 110, actuator assembly 120, and/or imaging system 130. Administration module 202 may comprise a user-configurable module, such that server 142 may store patient file data 230, images data 232, 3D model data 234, alignment data 236, biopsy plan data 238, tissue collection data 240, pathology data 242, treatment data 244, and stop criteria 246 either singularly or redundantly in database 144 or one or more other cloud systems or databases, according to particular needs.

According to embodiments, imaging module 204 configures, updates, and/or manages the operation of imaging system 130. Imaging module 204 may operate imaging system 130 and may store one or more tissue images generated by imaging system 130 in images data 232. In an embodiment, imaging module 204 is a user-configurable module, and may alter the operation of imaging system 130 in order to generate, for example, tissue images of varying degrees of contrast, depth, resolution, and/or spatial orientation. Imaging module 204 may generate tissue images and store tissue images in images data 232 substantially in real-time, at any periodic unit of time (such as, for example, every thirty seconds), or in response to input received by 3D mapping system 140 input device 146 (such as, for example, in response to the click of a mouse button).

Graphical user interface module 206 of server 142 may generate a graphical user interface, which 3D mapping system 140 may display on display device 148. Graphical user interface module 206 may generate a plurality of graphical user interfaces, depending on the information which the graphical user interface module 206 selects to display on display device 148, as described in greater detail below. Graphical user interface module 206 stores and retrieves data from database 144 including patient file data 230, images data 232, 3D model data 234, alignment data 236, biopsy plan data 238, tissue collection data 240, pathology data 242, and treatment data 244. By way of example only and not by way of limitation, graphical user interface module 206 may generate graphical user interface displays comprising one or more of tissue images stored in images data 232; tissue contours generated by contouring module 208 and stored in images data 232; one or more 3D tissue modules stored in 3D model data 234; 3D tissue model and imaging system 130 alignment information stored in alignment data 236; biopsy plan information stored in biopsy plan data 238; tissue specimen collection site information stored in tissue collection data 240; tissue lesion information stored in pathology data 242; and/or treatment plan information stored in treatment data 244.

According to embodiments, contouring module 208 assigns tissue structure contours to tissue images stored in images data 232. Tissue structure contours enable 3D mapping system 140 to identify specific tissue structures present in images data 232. Tissue structure contours may comprise, for example, whole organ contours (such as “prostate,” “kidney,” or any other organ), contours specific to a particular organ (such as “anterior lobe,” “transition zone,” or any other categorization), or contours applicable to any organ in a human or animal (such as “blood vessel”). Having assigned tissue structure contours to tissue images stored in images data 232, contouring module 208 stores the tissue structure contours associated with each tissue image in images data 232.

According to embodiments, model generation module 210 may access tissue images and tissue structure contours stored in images data 232. Model generation module 210 uses a sequence of tissue images and the tissue structure contours associated with each tissue image to generate a 3D tissue model of the organ or region illustrated by the tissue images. In an embodiment, the 3D tissue model may comprise one or more tissue subsections (such as a region designated “anterior lobe” on the 3D tissue model). According to embodiments, model generation module 210 may continually update the 3D tissue model based on real-time imaging system 130 information transmitted by the imaging module 204 and stored in images data 232. Model generation module 210 stores the 3D tissue model in 3D model data 234. Graphical user interface module 206 may access 3D model data 234, and may display the 3D tissue model on display device 148.

Alignment module 212 aligns the 3D tissue model with one or more real-time or stored tissue images. For example, in an embodiment, imaging module 204 may control imaging system 130 to generate a real-time tissue image, and may store data related to the real-time tissue image in images data 232. Graphical user interface module 206 may access images data 232 and 3D model data 234, and may display on display device 148 a 3D tissue model overlaid over the real-time tissue image. As described in greater detail below, alignment module 212 may align the tissue model with the real-time tissue image and confirm that one or more dimensions or tissue subsections of the 3D tissue model correspond to one or more dimensions or tissue structures of the real-time tissue image. Alignment module may store alignment data in alignment data 236.

Biopsy planning module 214 may access tissue images stored in images data 232, 3D tissue model data stored in 3D model data 234, and alignment data stored in alignment data 236. Biopsy planning module 214 may generate a biopsy plan specifying the sites and locations on the 3D tissue model from which needle assembly 110 will collect tissue specimens. Graphical user interface module 206 may display the biopsy plan on 3D mapping system 140 display device 148, and may superimpose the biopsy plan over the 3D tissue model and/or a real-time tissue image.

Biopsy planning module 214 may configure a number of biopsy plan parameters. By way of example only and not by way of limitation, biopsy planning module 214 may alter the number of tissue specimens that will be collected during the biopsy, the size of the cannula and mandrel 114 used by needle assembly 110 to collect tissue specimens for biopsy and the according diameter of the tissue specimen collected, the three-dimensional spacing between tissue specimen collection sites, the length of the tissue specimen that needle assembly 110 will collect at a particular tissue specimen collection site, and/or any other biopsy plan parameter. Biopsy planning module 214 may configure biopsy plan parameters in response to input received through 3D mapping system 140 input device 146, and may automatically adjust one biopsy plan parameter (such as, for example, the three-dimensional spacing between tissue specimen collection sites) in response to changes made to another biopsy plan parameter (such as, for example, the size of needle assembly 110 cannula and mandrel 114 used to collect tissue specimens). In an embodiment, graphical user interface module 206 displays the current form of the biopsy plan on display device 148, and updates the biopsy plan displayed on display device 148 to reflect all biopsy plan parameter alterations input by input device 146, 3D mapping system 140, and/or system 100. Biopsy planning module 214 may store the biopsy plan, and changes or updates made to the biopsy plan, in biopsy plan data 238.

According to embodiments, tissue collection module 216 configures, updates, and/or manages the operation of needle assembly 110 and actuator assembly 120. Tissue collection module 216 may operate actuator needle assembly 110 and actuator assembly 120, and may control the collection of one or more tissue specimens. In an embodiment, tissue collection module 216 may access biopsy plan data 238, and may actuate actuator assembly 120 to extend one or more needle assemblies 110 into patient 160 at one of the sites specified by the biopsy plan. Imaging module 204 may control imaging system 130 to generate a substantially real-time tissue image comprising the location of one or more needle assemblies 110 extended into patient 160, and may store the substantially real-time tissue image in images data 232. Graphical user interface module 206 may access images data 232, 3D model data 234, alignment data 236, and biopsy plan data 238, and may display on display device 148 the substantially real-time tissue image comprising the location of the one or more needle assembles overlaid over and aligned with the 3D tissue model and biopsy plan. Tissue collection module 216 may access images data 232, 3D model data 234, alignment data 236, and biopsy plan data 238 to confirm that actuator assembly 120 extended one or more needle assembles 110 into the correct tissue locations of patient 160 specified by the biopsy plan. Tissue collection module 216 may store the alignment coordinates from which needle assembly 110 collected each tissue specimen in tissue collection data 240.

In an embodiment, pathology module 218 assigns pathology results to each of the one or more tissue specimens stored in tissue collection data 240. By way of example only and not by way of limitation, the tissue specimens collected by tissue collection module 216 and one or more needle assemblies 110 are biopsied by a medical professional, such as a urologist, to locate the presence of lesions, cancer, or any other form of tissue. In an embodiment, a medical professional, such as a pathologist, or an embodiment (such as, for example, a machine) capable of rendering tissue information electronically, examines each tissue specimen in its entirety or in part, collected by tissue collection module 216 executing the actions of method 900, described in greater detail below. The medical professional stores the pathology results of each tissue specimen biopsy in pathology data 242. Pathology module 218 accesses pathology data 242 and stores tissue specimen test results and remarks regarding each separate tissue specimen in tissue collection data 240. In an embodiment, pathology module 218 may receive input from input device 146, such as, for example, input specifying the Gleason score or grade or other determination of cancer or tissue type result of a particular tissue specimen, which pathology module 218 may store in tissue collection data 240. In another embodiment, pathology module 218 may access biopsy information for each tissue specimen stored in a cloud system or separate database and which is connected to pathology module 218 by network 150, and may store the biopsy information for each tissue specimen in tissue collection data 240. In an embodiment, pathology module 218 may generate a pathology visualization interface and treatment plan to treat one or more lesions, cancer, or other tissue anomalies, as described in greater detail below. Pathology module 218 may store the treatment plan in treatment data 244.

In an embodiment, treatment module 220 may update the treatment plan stored in treatment data 244 in response to the actions of a medical professional treating one or more tissues according to the treatment plan, as described in greater detail below. By way of example only and not by way of limitation, treatment module 220 may store in treatment plan data 244 and alignment data 236 the alignment coordinates of tissue successfully treated by the medical professional.

Patient file data 230 of database 144 may comprise medical history data of patient 160. Patient file data 230 may comprise, for example, the name, address, and previous medical history of patient 160. Patient file data 230 may also comprise a patient's previously-generated tissue images, tissue structure contours, 3D tissue models, and/or biopsy plans.

Images data 232 may comprise one or more tissue images generated by imaging module 204. Imaging module 204 may store tissue images in images data 232 in any format, including but not limited to individual image files (such as, for example, JPEG, PNG, or TIFF images) or video files (such as in MOV format). Imaging module 204 may retrieve patient name or other identifying data from patient file data 230, and may store the patient name or other identifying data associating each tissue image with a particular patient in images data 232.

3D model data 234 comprises one or more 3D tissue models generated by model generation module 210. Model generation module 210 may store 3D tissue models in 3D model data 234 in any format, including but not limited to STL (stereolithography), OBJ, FBX, COLLADA, and/or 3DS formats. Model generation module 210 may retrieve patient name or other identifying data from patient file data 230, and may store the patient name or other identifying data associating each 3D tissue model with a particular patient in 3D model data 234.

Alignment data 236 comprises alignment data that associates each tissue image stored in images data 232 with one or more 3D tissue models stored in 3D model data 234. By way of example only and not by way of limitation, alignment module may divide each 2D image and/or each 3D tissue model into alignment coordinates, specifying the distances between tissue structure contours or other components on each 2D image and/or each 3D tissue model. 3D mapping system 140 may use alignment coordinates to, for example, superimpose a 2D tissue image over a 3D tissue model, and to align the tissue structure contours of the 2D tissue image that match with the tissue structure contours of the 3D tissue model, as described in greater detail below. Alignment data 236 may comprise alignment coordinates measured in millimeters, imaging system 130 coordinates, 3D tissue model coordinates, or any other unit of measurement.

According to embodiments, and as described in greater detail below, biopsy plan data 238 may comprise any data that specifies one or more parameters of the biopsy plan generated by biopsy planning module 214.

According to embodiments, and as described in greater detail below, biopsy plan tissue collection data 240 may comprise any data pertaining to one or more tissue specimens collected by tissue collection module 216. Tissue collection data 240 may comprise, for example, alignment coordinates from which tissue collection module 216 used needle assembly 110 to collect a particular tissue specimen.

According to embodiments, and as described in greater detail below, pathology data 242 may comprise pathology results for one or more tissue specimens. In an embodiment, a medical professional examines and biopsies tissue specimens collected by tissue collection module 216 at action 304. The medical professional stores the results of each tissue specimen biopsy in pathology data 242. Pathology data 242 may comprise, for example, tissue Gleason scores, lesion sizes, pathology notes, and/or any other pathology information pertaining to one or more tissue samples.

According to embodiments, and as described in greater detail below, treatment data 244 comprises one or more tissue treatment plans generated by pathology module 218.

Stop criteria 246 may comprise data instructing imaging module 204 to cease generating tissue images and storing tissue images in images data 232. As described in greater detail below, model generation module 210 generates one or more 3D tissue models by accessing a sequence of tissue images and the tissue structure contours associated with each tissue image stored in images data 232. In an embodiment, a greater number of tissue images and/or tissue structure contours may permit model generation module 210 to generate a 3D tissue model of higher resolution or fidelity. Stop criteria 246 specifies the number of tissue images imaging module 204 should generate and store in images data 232 before model generation module 210 generates one or more 3D tissue models. In an embodiment, stop criteria 246 comprises a discrete number of tissue images (such as, for example, ten tissue images). In other embodiments, stop criteria 246 may comprise input from input device 146 instructing imaging module 204 to cease generating tissue images.

In an embodiment, and as described in greater detail below, stop criteria 246 may comprise criteria that determine whether tissue collection module 216 modifies one or more biopsy plan parameters after extending needle assembly 110 into a region of tissue that does not correspond to a tissue collection site specified by the biopsy plan.

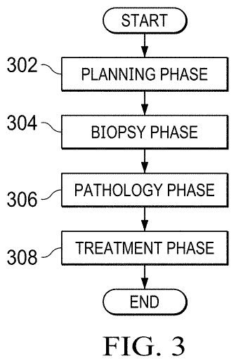

FIG. 3 illustrates exemplary method 300 of imaging, planning, and conducting a tissue biopsy, developing a treatment plan, and implementing a treatment plan, according to an embodiment. In an embodiment, 3D mapping system 140 operates imaging system 130 to collect tissue images of human or animal patient 160, generates a 3D tissue model of the imaged tissue, generates a biopsy plan to collect one or more tissue specimens from one or more designated locations on the 3D tissue model, and operates actuator assembly 120 and needle assembly 110 to collect one or more tissue specimens according to the biopsy plan. 3D mapping system 140 displays the tissue specimen biopsy results on the 3D tissue model, and generates a treatment plan to treat lesions, cancer, or tissue anomalies located during the biopsy. Although actions of method 300 are described in a particular order, embodiments contemplate actions performed in any suitable order or combination according to particular needs.

Action 302 comprises a biopsy planning phase. At action 302, and according to embodiments, 3D mapping system 140 operates imaging system 130 and acquires one or more tissue images from human or animal patient 160. Contouring module 208 assigns tissue structure contours to the one or more tissue images. Model generation module 210 accesses the one or more tissue images and tissue structure contours assigned to each tissue image, and generates a 3D tissue model of the organ or region illustrated by the tissue images. Graphical user interface module 206 displays the 3D tissue model, overlaid with a real-time or stored tissue image generated by imaging module 204, on 3D mapping system 140 display device 148. Alignment module 212 aligns the 3D tissue model with the real-time or stored tissue image using alignment coordinates stored in alignment data 236. Biopsy planning module 214 generates and modifies a biopsy plan, specifying the sites and locations on the 3D tissue model from which needle assembly 110 will collect tissue specimens. Graphical user interface module 206 displays the biopsy plan, and any adjustments or modifications biopsy planning module 214 makes to the biopsy plan, on display device 148.

Action 304 comprises a biopsy phase. At action 304, and according to embodiments, tissue collection module 216 accesses the biopsy plan and selects a first tissue specimen collection site specified by the biopsy plan. Tissue collection module 216 actuates actuator assembly 120 to extend needle assembly 110 into patient 160 at the first tissue specimen site specified by the biopsy plan. Imaging module 204 operates imaging system 130, and generates a real-time tissue image displaying the location of needle assembly 110 in patient 160. Graphical user interface module 206 displays, on display device 148, the real-time tissue image displaying needle assembly 110 location. Tissue collection module 216 updates the alignment coordinates of needle assembly 110 by comparing the location of needle assembly 110 on the real-time tissue images with the intended alignment coordinate location of the tissue specimen collection site specified by the biopsy plan. In the event actuator assembly 120 has extended needle assembly 110 into a segment of tissue that the biopsy plan did not specify as a tissue specimen collection site, tissue collection module 216 either modifies the location of the biopsy plan tissue specimen collection site to match the actual location of needle assembly 110, modifies the biopsy plan in another manner as described in greater detail below, or retracts needle assembly 110 for reinsertion into the correct tissue region.

Continuing action 304, and according to embodiments, if tissue collection module 216 determines actuator assembly 120 extended needle assembly 110 into the correct tissue specimen collection site specified by the biopsy plan, or if tissue collection module 216 modifies the location of the biopsy plan tissue specimen collection site to match the actual location of needle assembly 110, tissue collection module 216 extracts a tissue specimen for biopsy and analysis. Tissue collection module 216 stores, in tissue collection data 240, alignment coordinates describing the location in the tissue of patient 160 from which tissue collection module 216 extracted the tissue specimen. Tissue collection module 216 actuates actuator assembly 120 to retract needle assembly 110 containing the tissue specimen. Tissue collection module 216 stores each tissue specimen in actuator assembly 120 for biopsy and analysis.

Continuing action 304, and according to embodiments, tissue collection module 216 accesses the biopsy plan and selects the next tissue specimen collection site specified by the biopsy plan. Tissue collection module 216 continues operating in the manner described above to collect the remaining tissue specimens indicated by the biopsy plan. Throughout this process, and as described in greater detail below, tissue collection module 216 may modify the biopsy plan by adding additional tissue specimen collection sites to the biopsy plan, subtracting tissue specimen collection sites from the biopsy plan, modifying the location of one or more biopsy plan tissue specimen collection sites, and/or adjusting the biopsy plan in response to input from input device 146 (such as, for example, mouse button clicks adding additional tissue specimen collection sites to the biopsy plan).

Action 306 comprises a pathology phase. At action 306, and according to embodiments, a medical professional biopsies each tissue specimen collected by tissue collection module 216 at action 304. The medical professional stores the results of each tissue specimen biopsy in pathology data 242. Pathology module 218 accesses 3D model data 234 and pathology data 242, and displays on the 3D tissue model the biopsy results of each of the tissue specimen collection sites specified by the biopsy plan, including the location of any lesions, cancers, or tissue anomalies present. Pathology module 218 stores the 3D tissue model including the biopsy results of each of the tissue specimen collection sites in 3D model data 234.

Continuing action 306, and according to embodiments, graphical user interface module 206 accesses the 3D tissue model stored in 3D model data 234, and displays on display device 148 the 3D tissue model with lesions, cancers, or tissue anomalies. Pathology module 218 generates a treatment plan to treat the lesions, cancers, or tissue anomalies.

Action 308 comprises a treatment phase. In an embodiment, 3D mapping system 140 acquires a real-time tissue image of patient 160. Treatment module accesses the treatment plan, and updates the treatment plan as a medical professional treats one or more patient 1660 tissue regions according to the treatment plan.

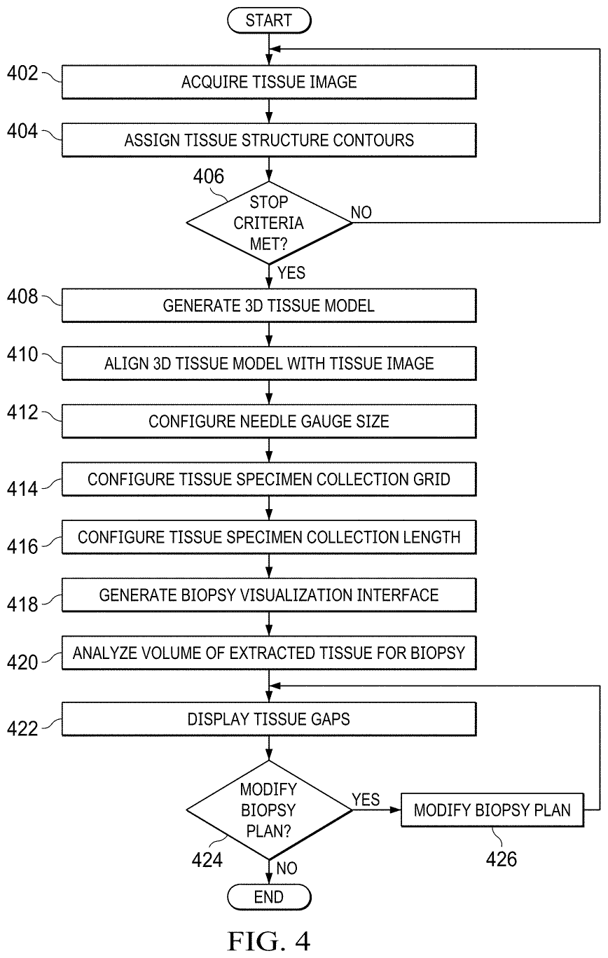

FIG. 4 illustrates exemplary method 400 of conducting a biopsy planning phase, according to an embodiment. In an embodiment, 3D mapping system 140 performs the actions of method 400 to image the tissue of patient 160, create a 3D tissue model, and generate and modify a biopsy plan. Although actions of method 400 are described in a particular order, embodiments contemplate actions performed in any suitable order or combination according to particular needs.

At action 402, imaging module 204 acquires a tissue image from patient 160. In an embodiment, imaging module 204 operates imaging system 130 and stores a tissue image generated by imaging system 130 in images data 232. Imaging module 204 may control the operation of imaging system 130 and may change imaging system 130 parameters to generate, for example, tissue images of varying degrees of contrast, depth, resolution, and/or spatial orientation. Graphical user interface module 206 may display on display device 148 a real-time tissue image generated by imaging module 204 to aid in the selection of a particular tissue image to store in images data 232. Imaging module 204 may modify the operation of imaging system 130, and the resulting tissue images generated by imaging system 130, in response to one or more inputs received by input device 146.

To provide an example of 3D mapping system 140 executing the actions of method 400 and not by way of limitation, imaging module 204 at action 402 operates imaging system 130 to generate a real-time tissue image of a prostate of human patient 160. In other embodiments, 3D mapping system 140 may execute the actions of method 400 to generate a real-time image of any other tissue or organ, according to particular needs. Graphical user interface module 206 displays the real-time prostate tissue image on display device 148. Imaging module 204 adjusts imaging system 130 parameters to increase the tissue image contrast between light and dark sections of the tissue image. Image module 204 saves and stores the resulting prostate tissue image in images data 232, and associates the tissue image with the name and medical history of patient 160 stored in patient file data 230. In this embodiment, imaging module 204 stores in images data 232 spatial location and orientation data that indicates the alignment coordinates in 3D space from which imaging module 204 generated each tissue image, as described in greater detail below.

At action 404, contouring module 208 assigns tissue structure contours to the tissue image stored in images data 232. Tissue structure contours identify specific tissue structures present in the tissue image stored in images data 232. In an embodiment, contouring module 208 accesses the tissue image stored in images data 232, automatically detects tissue structures visible in the tissue image, and assigns tissue structure contours to the tissue structures visible in the tissue image. In this embodiment, contouring module 208 assigns tissue structure contours from a database of possible tissue structure contours (such as, for example, “prostate boundary” or “anterior lobe”) stored in database 144. In other embodiments, contouring module 208 accepts input from input device 146 to allow a medical professional to define and trace the contour outlines of one or more tissue structures visible in the tissue image.

In an embodiment, graphical user interface module 206 accesses the tissue image stored in images data 232 and displays the tissue image on display device 148. Contouring module 208 overlays a tracing cursor over the tissue image displayed on display device 148. Contouring module 208 moves the tracing cursor in response to input received by input device 146, and assigns tissue structure contours to tissue image regions or structures traced by the tracing cursor, as described in greater detail below.

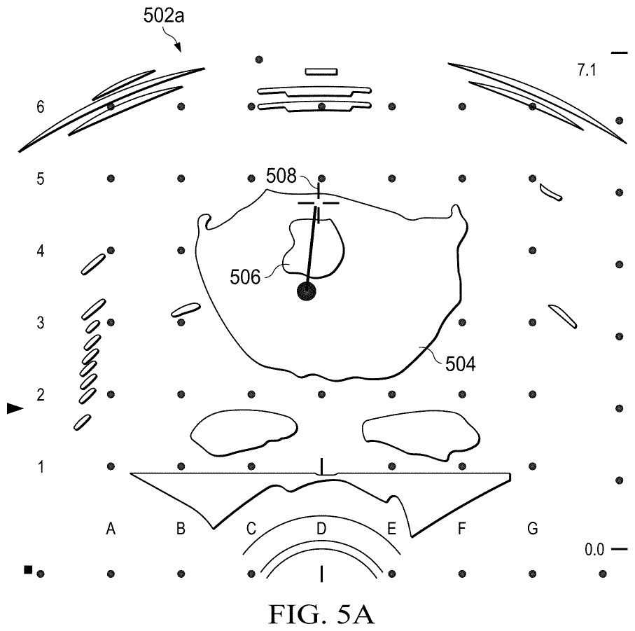

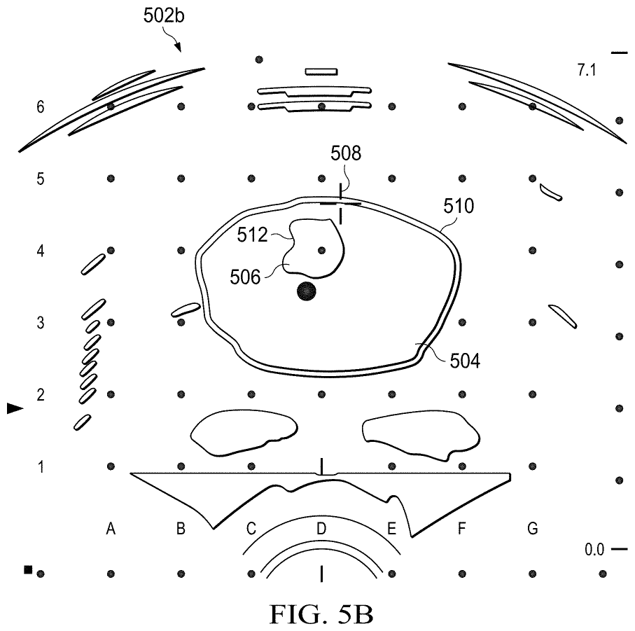

FIGS. 5A-5B illustrates contouring visualization interfaces 502a and 502b, according to embodiments. In an embodiment, contouring visualization interfaces 502a and 502b may comprise prostate tissue 504, urethra 506, tracing cursor 508, prostate boundary contour 510, and urethra boundary contour 512. Although contouring visualization interfaces 502a and 502b, prostate tissue 504, urethra 506, tracing cursor 508, prostate boundary contour 510, and urethra boundary contour 512 are shown and described in an exemplary configuration, embodiments contemplate any number or configuration of contouring visualization interfaces 502a and 502b, prostate tissues 504 or any other tissues or tissue structures, urethras 506, tracing cursors 508, prostate boundary contours 510, urethra boundary contours 512, or any other tissue boundary contours, according to particular needs. Although FIGS. 5A-5B illustrate contouring visualization interfaces 502a and 502b with respect to prostate tissue 504, including nearby tissue structures such as urethra 506, embodiments contemplate generating contouring visualization interfaces 502 to contour the structures of any tissue, organ, suspected or confirmed region of cancer, or anatomical structure of any human or animal, according to particular needs.

Continuing with the above example, graphical user interface module 206 accesses the prostate tissue image stored in images data 232 that imaging module 204 generated at action 402. Graphical user interface module 206 generates contouring visualization interface 502 displaying the prostate tissue image, and displays contouring visualization interface 502 on display device 148. Contouring visualization interface 502 displays prostate tissue 504 in the center of contouring visualization interface 502.

Continuing the example, contouring module 208 overlays tracing cursor 508 over prostate tissue 504 on contouring visualization interface 502. Contouring module 208 moves the location of tracing cursor 508 on contouring visualization interface 502 in response to input received from input device 146. In this example, a medical professional (not illustrated in FIGS. 5A-5B) uses input device 146 to move tracing cursor 508 around the outline of prostate tissue 504 on contouring visualization interface 502. As best illustrated by FIG. 5B, the medical professional traces prostate boundary contour 510 around prostate tissue 504. Contouring module 208 designates prostate boundary contour 510 as the boundary between relevant (prostate) and non-relevant (surrounding) tissue on the prostate tissue image. In a similar fashion, the medical professional traces urethra boundary contour 512 around urethra 506 to delineate the boundary between prostate tissue 504 and urethra 506 on contouring visualization interface 502. Contouring module 208 stores prostate boundary contour 510 and urethra boundary contour 512 in images data 232.

At action 406, imaging module 204 accesses stop criteria 246 to determine whether one or more stop criteria have been met. As described above, stop criteria 246 may specify the number of tissue images imaging module 204 should generate and store in images data 232 before model generation module 210 generates a 3D tissue model from the tissue images and tissue structure contours stored in images data 232. If imaging module 204 determines no stop criteria 246 have been met (such as, for example, in an embodiment in which stop criteria 246 specify that imaging module 204 should generate and store in images data 232 50 tissue images, and imaging module 204 has currently generated and stored in images data 232 only 25 tissue images), imaging module 204 returns to action 402 and continues generating tissue images and storing tissue images in images data 232, and contouring module 208 continues, at action 404, assigning tissue structure contours to issue images stored in images data 232, until imaging module 204 determines one or more stop criteria 246 are met. If imaging module 204 determines one or more stop criteria 246 have been met, model generation module 210 executes action 408, as described in greater detail below.

Continuing the example, at action 406, imaging module 204 accesses stop criteria 246. In the particular embodiment illustrated by this example, stop criteria 246 specifies that imaging module 204 will generate 20 prostate tissue images. In other embodiments, stop criteria 246 may specify that imaging module 204 will generate 5, 10, 50, or any other number of tissue images. Imaging module 204 and contouring module 208 continue to execute actions 402, 404, and 406 described above, until imaging module 204 determines at action 406 that it has generated 20 prostate tissue images and stop criteria 246 is activated.

At action 408, model generation module 210 accesses images data 232 and generates a 3D tissue model based on the tissue images and tissue structure contours stored in images data 232. In an embodiment, model generation module 210 arranges the tissue images and tissue structure contours in a sequential manner and interpolates, using the location of tissue structure contours present in each tissue image, 3D tissue structures that model generation module 210 uses to generate the 3D tissue model. Imaging module 204 may store in images data 232 spatial location and orientation data that indicates the alignment coordinates in 3D space from which imaging module 204 generated each tissue image. Model generation module 210 may access the spatial location and orientation data assigned to each tissue image stored in images data 232, and may use the spatial location and orientation data to arrange the tissue images with respect to one another in 3D space (for example, by placing one tissue image 2 centimeters anterior to another tissue image in 3D space). Model generation module 210 joins the tissue contour structures stored in each tissue image to create a 3D tissue model, and stores the 3D tissue model in 3D model data 234.

Continuing the example, model generation module 210 accesses the 20 prostate tissue images stored in images data 232. Each prostate tissue image stored in images data 232 further comprises prostate boundary contour 510 and urethra boundary contour 512. Model generation module 210 arranges the 20 prostate tissue images according to the special location and orientation data that indicates the alignment coordinates in 3D space from which imaging module 204 generated each prostate tissue image. Model generation module 210 joins prostate boundary contour 510 and urethra boundary contour 512 of each prostate tissue image to create a 3D prostate model. Model generation module 210 stores the 3D prostate model in 3D model data 234.

At action 410, and according to embodiments, alignment module 212 accesses 3D model data 234, and aligns the 3D tissue model with one or more real-time or stored tissue images. By confirming the 3D tissue model generated by model generation module 210 conforms to one or more real-time or stored tissue images, alignment module 212 enables 3D mapping system 140 to display the location of one or more needle assemblies 110 extended into patient 160 on the 3D tissue model, and to confirm that one or more needle assemblies 110 collect tissue specimens from the correct tissue location as specified by the 3D tissue model and biopsy plan, as described in greater detail below.

According to embodiments, alignment module 212 aligns the 3D tissue model with a real-time tissue image or one or more tissue images stored in images data 232. By way of example only and not by way of limitation, at action 410, graphical user interface module 206 may access images data 232, and may display on display device 148 one or more tissue images and the tissue structure contours associated with one or more tissue images. Alignment module 212 may access 3D model data 234 and may superimpose the 3D tissue model over the one or more tissue images on display device 148. Alignment module 212 may increase or decrease the size of the 3D tissue model or adjust the position or orientation of the 3D tissue model to correspond with one or more tissue structure contours which contouring module 208 associated with the one or more tissue images. Having aligned the 3D tissue model with the one or more tissue images, alignment module 212 stores alignment coordinates matching the tissue image to the 3D tissue model in alignment data 236.

In another embodiment, and at action 410, imaging module 204 may control imaging system 130 to generate a real-time tissue image of the tissue of patient 160. Graphical user interface module 206 may display the real-time tissue image on display device 148. Alignment module 212 may access 3D model data 234 and may superimpose the 3D tissue model over the real-time tissue image on display device 148. Alignment module 212 may accept input from input device 146 (such as, for example, input from a medical professional aligning the 3D tissue model with the real-time tissue image) to increase or decrease the size of the 3D tissue model or adjust the position or orientation of the 3D tissue model to correspond with the superimposed real-time tissue image. Having aligned the 3D tissue model with the real-time tissue image, alignment module 212 stores alignment coordinates matching the tissue image to the 3D tissue model in alignment data 236.

Continuing the example, at action 410, imaging module 204 controls imaging system 130 to generate a real-time prostate tissue image of patient 160 prostate tissue 504. Graphical user interface module 206 displays the real-time prostate tissue image on display device 148. Alignment module 212 accesses 3D model data 234 and superimposes the 3D prostate model over the real-time prostate tissue image on display device 148. Alignment module 212 accepts input from input device 146 (comprising, in this example, input from a medical professional aligning the 3D prostate model with the real-time prostate tissue image) to increase the size of the 3D prostate model and to adjust the position and orientation of the 3D prostate model to correspond with the superimposed real-time prostate tissue image. Having aligned the 3D prostate model with the real-time prostate tissue image, alignment module 212 stores alignment coordinates matching the 3D prostate model to the real-time prostate tissue image in alignment data 236.

At actions 412-426 and as described in greater detail below, biopsy planning module 214 generates a biopsy plan that specifies on the 3D tissue model (or, according to embodiments, on a 2D tissue model) the locations from which needle assembly 110 will collect tissue specimens at action 304 of method 300. According to embodiments, biopsy planning module 214 may access tissue images stored in images data 232, 3D tissue model data stored in 3D model data 234, and alignment data stored in alignment data 236. Biopsy planning module 214 may generate a biopsy plan specifying, with alignment coordinates stored in alignment data 236, the sites and locations on the 3D tissue model from which needle assembly 110 will collect tissue specimens. Graphical user interface module 206 may display the biopsy plan on 3D mapping system 140 display device 148, and may superimpose the biopsy plan over the 3D tissue model and/or a real-time tissue image or a tissue image stored in images data 232, as described in greater detail below.

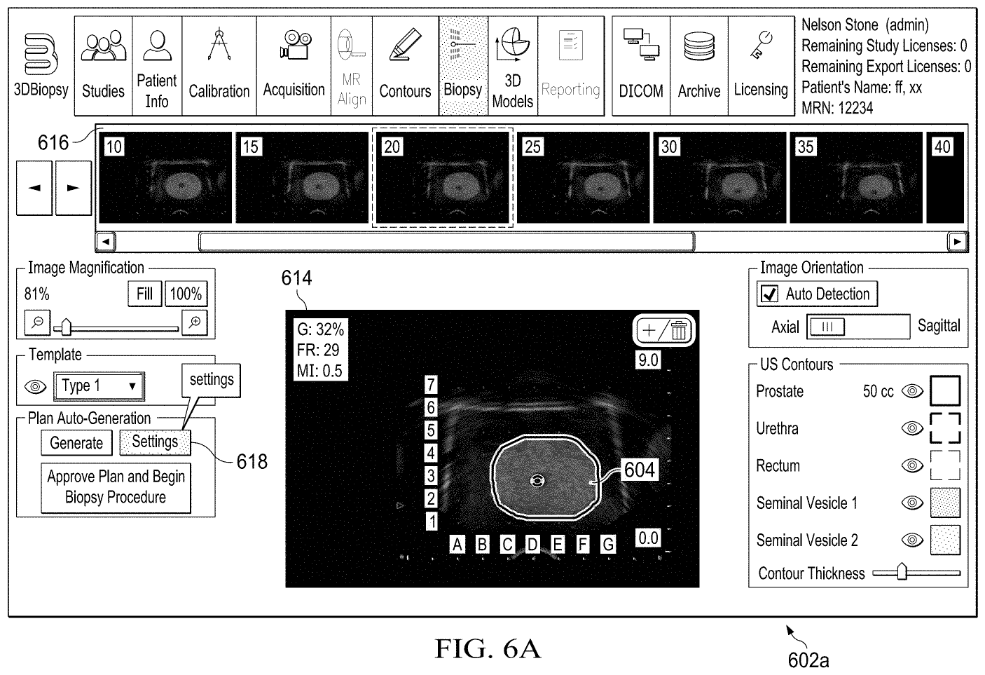

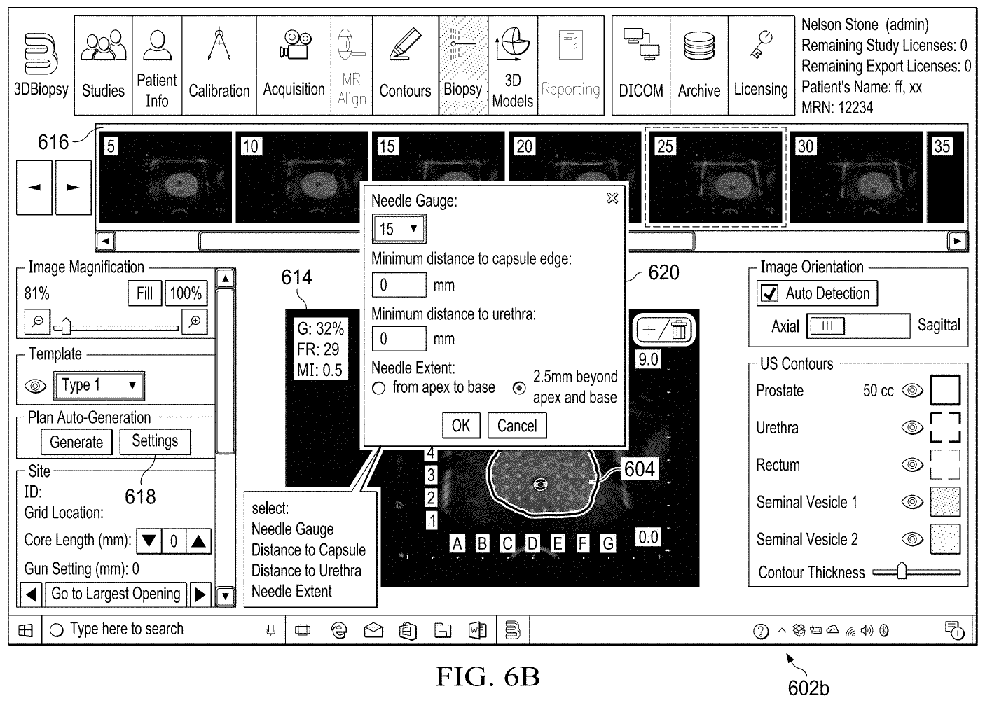

FIGS. 6A-6B illustrate exemplary biopsy planning interfaces 602a and 602b, according to embodiments. In an embodiment, biopsy planning interface 602 may comprise tissue image display 614 displaying prostate tissue 604, tissue image scroll box 616, biopsy settings button 618, and biopsy settings dialogue box 620. Although biopsy planning interface 602, tissue image display 614, prostate tissue 604, tissue image scroll box 616, biopsy settings button 618, and biopsy settings dialogue box 620 are shown and described in an exemplary configuration, embodiments contemplate any number or configuration of biopsy planning interfaces 602, tissue image displays 614, prostate tissues 604 or any other tissues displayed by tissue image displays 614, tissue image scroll boxes 616, biopsy settings buttons 618, biopsy settings dialogue boxes 620, and/or other biopsy planning interface 602 features, displays, tissues, boxes, or buttons, according to particular needs.

In an embodiment, biopsy planning module 214 accesses tissue images stored in images data 232, 3D tissue model data stored in 3D model data 234, and alignment data stored in alignment data 236, and generates biopsy planning interface 602. Biopsy planning module 214 may store biopsy planning interface 602 in biopsy plan data 238. Graphical user interface module 206 accesses biopsy plan data 238 and displays biopsy planning interface 602 on display device 148.

According to embodiments, biopsy planning interface 602 comprises tissue image display 614. Tissue image display 614 displays one or more real-time tissue images, such as, for example, a real-time image of prostate tissue 604, generated by imaging module 204, or one or more stored tissue images saved in images data 232. According to embodiments, biopsy planning interface 602 comprises tissue image scroll box 616. Tissue image scroll box 616 displays a subset of the tissue images stored in images data 232. Biopsy planning module 214 may pan tissue image scroll box 616 left and right, thereby displaying different tissue images in tissue image scroll box 616, in response to input received through input device 146.

According to embodiments, biopsy planning interface 602 comprises biopsy settings button 618. Biopsy settings button 618 may open biopsy settings dialogue box 620 in response to input received from input device 146. In an embodiment, biopsy planning module 214 may overlay a graphical user interface cursor (not shown in FIGS. 6A and 6B) on biopsy planning interface 602. Biopsy planning module 214 may move the graphical user interface cursor on biopsy planning interface 602 in response to input received from input device 146. In this embodiment, and in response to input device 146 moving the graphical user interface cursor over biopsy settings button 618 and activating biopsy settings button 618 (such as, for example, in response to a medical professional moving a computer mouse to move the graphical user interface cursor over biopsy settings button 618, and ‘clicking’ a computer mouse button), biopsy planning module 214 may display biopsy settings dialogue box 620 on biopsy planning interface 602.

According to embodiments, biopsy settings dialogue box 620 permits biopsy planning module 214 to configure several biopsy plan settings. In an embodiment, biopsy settings dialogue box 620 configures the number of tissue specimens that will be collected during the biopsy, the size of the cannula and mandrel 114 used by needle assembly 110 to collect tissue specimens for biopsy and the according diameter of the tissue specimen collected, the minimum distance between tissue specimen collection sites, the specific location as measured in alignment coordinates of one or more tissue specimen collection sites, the length of the tissue specimen that needle assembly 110 will collect at a particular tissue specimen collection site, and/or any other biopsy plan parameter. In an embodiment, biopsy planning module 214 may configure biopsy parameters displayed in biopsy settings dialogue box 620 (such as, for example, the size in needle gauge of the needle cannula and mandrel 114 used by needle assembly 110) in response to input received by input device 146.

At action 412, and according to embodiments, biopsy planning module 214 configures the needle gauge size of needle assembly 110 that will extract tissue specimens from the tissue specimen collection sites specified by the biopsy plan. In an embodiment, biopsy planning module 214 configures the needle gauge size in response to input that biopsy settings dialogue box 620 receives from input device 146. In other embodiments, at action 412, biopsy planning module 214 automatically chooses the needle gauge size of needle assembly 110. In an embodiment, biopsy planning module 214 may select, for example, a needle gauge size from needles of 15, 17, or 18 gauge diameters. Having configured the needle gauge size of needle assembly 110, biopsy planning module 214 stores the selected needle gauge size in biopsy plan data 238.

Continuing the example, at action 412, biopsy planning module 214 accesses tissue images stored in images data 232, 3D tissue model data stored in 3D model data 234, and alignment data stored in alignment data 236, and generates biopsy planning interface 602. Graphical user interface module 206 accesses biopsy plan data 238 and displays biopsy planning interface 602 on display device 148. In this example, biopsy planning interface 602 comprises tissue image display 614, prostate tissue 604, tissue image scroll box 616, and biopsy settings button 618, as best illustrated in FIG. 6A.

Continuing the example, at action 412, biopsy planning module 214 overlays a graphical user interface cursor on biopsy planning interface 602. Biopsy planning module 214 moves the graphical user interface cursor on biopsy planning interface 602 in response to input received from input device 146. In this example, a medical professional uses a computer mouse as input device 146 to move the graphical user interface cursor over biopsy settings button 618. The medical professional clicks biopsy settings button 618. In response, biopsy planning module 214 opens biopsy settings dialogue box 620 on biopsy planning interface 602 (best illustrated in FIG. 6B), and uses input device 146 (in this example, a keypad) to configure the biopsy plan and needle assembly 110 to use an 18 gauge needle. Biopsy planning module 214 stores the selection of an 18 gauge needle in biopsy plan data 238.

At action 414, and according to embodiments, biopsy planning module 214 configures the tissue specimen collection grid of biopsy plan tissue specimen collection sites. In an embodiment, and as described in greater detail below, 3D mapping system 140 is configured to operate actuator assembly 120 and needle assembly 110 to extract tissue specimens from a rectangular tissue specimen collection grid of tissue specimen collection sites. The rectangular tissue specimen collection grid may permit 3D mapping system 140 to collect and biopsy tissue specimens from (1) a tissue location that is determined to contain a tissue lesion, cancer, or other tissue anomaly, and (2) the tissue locations immediately surrounding the lesion. 3D mapping system 140 may use the biopsy information provided by collecting and biopsying tissue specimens in a rectangular tissue specimen collection grid pattern to visualize lesions, cancer, or other tissue anomalies with a high degree of accuracy and precision, and to generate treatment plans in response, as described in greater detail below.

In an embodiment, at action 414, biopsy planning module 214 may configure the tissue specimen collection grid by automatically selecting a minimum distance between tissue specimen collection sites. Biopsy planning module 214 may specify minimum distances between tissue specimen collection sites as measured in millimeters, alignment coordinates and/or alignment data 236, or in any other format. In other embodiments, biopsy planning module 214 generates biopsy settings dialogue box 620 in the manner described above, and biopsy settings dialogue box 620 receives input from input device 146 specifying minimum distances between tissue specimen collection sites. Having configured the tissue specimen collection grid, biopsy planning module 214 generates alignment coordinates for each of the tissue specimen collection sites on the 3D tissue model, and stores the configured tissue specimen collection grid and alignment coordinates in biopsy plan data 238.

Continuing the example, at action 414, biopsy planning module 214 configures the tissue specimen collection grid. In this example, biopsy planning interface 602, including biopsy settings dialogue box 620, remains displayed on display device 148 from action 412. The medical professional uses input device 146 to enter, into biopsy settings dialogue box 620, 4 millimeters as a minimum distance between tissue specimen collection sites. Biopsy planning module 214 generates alignment coordinates for each of the tissue specimen collection sites on the 3D prostate model, and stores the configured specimen collection grid and alignment coordinates in biopsy plan data 238.

At action 416, and according to embodiments, biopsy planning module 214 configures the tissue specimen length that needle assembly 110 will collect at action 304 of method 300. According to embodiments, mandrel 114 of needle assembly 100 may comprise configurable core bed 116 to permit the extraction of tissue specimens of variable length, up to and including the length of mandrel 114 itself, as measured along the long axis of needle assembly 110. In an embodiment, needle assembly 100 may collect a single, continuous tissue specimen that runs from one edge of a target tissue structure to the opposite edge of the target tissue structure along the axis of the needle's insertion. In other embodiments, needle assembly 100 may collect a tissue specimen that is, for example, 0.5 centimeters long, 1 centimeter long, or any other length.

In an embodiment, at action 416, biopsy planning module 214 may configure the tissue specimen length by automatically selecting a tissue specimen length (such as, for example, 2 centimeters). In other embodiments, biopsy planning module 214 generates biopsy settings dialogue box 620 in the manner described above, and biopsy settings dialogue box 620 receives input from input device 146 specifying the tissue specimen length. Having configured the tissue specimen length, biopsy planning module 214 stores the configured tissue specimen length in biopsy plan data 238.

Biopsy planning module 214 may automatically adjust tissue specimen lengths for tissue specimen collection sites that are not long enough to accommodate the collection of a full-length tissue specimen at the particular tissue specimen collection site. By way of example only and not by way of limitation, in an embodiment in which biopsy planning module 214 generates a biopsy plan to collect a rectangular grid of tissue specimens from human prostate tissue, and biopsy planning module has selected 2 centimeters as a tissue specimen length for needle assembly 110, only tissue specimen collection sites near the center of the prostate will comprise sufficient length to permit the extraction of a 2 centimeter tissue specimen. Tissue specimen collection sites near the edge of the prostate tissue may only permit much smaller tissue specimen lengths (such as, for example, 0.1 centimeters near the edge of the prostate tissue) without breaking through the prostate tissue boundary and collecting a tissue specimen from non-prostate tissue. In this exemplary embodiment, biopsy planning module 214 automatically detects on the 3D tissue model tissue specimen collection sites that will not accommodate the full length of the selected tissue specimen length. Biopsy planning module 214 shortens the tissue specimen lengths needle assembly 110 will remove from the tissue specimen collection sites located near the edge of the prostate tissue to avoid breaking through the prostate tissue boundary and collecting tissue specimens from non-prostate tissue.

Continuing the above example, at action 412, biopsy planning module 214 configures the tissue specimen length that needle assembly 100 will collect during the upcoming prostate biopsy. In this example, biopsy planning interface 602, including biopsy settings dialogue box 620, remains displayed on display device 148 from action 412. The medical professional uses input device 146 to enter, into biopsy settings dialogue box 620, 4 centimeters as the tissue specimen length. Biopsy planning module 214 automatically shortens the tissue specimen length for tissue specimen collection sites near the edge of the 3D prostate model (or, according to embodiments, near the edge of a 2D prostate model) that cannot accommodate, for example, a full 4 centimeter tissue specimen length, and stores the configured tissue specimen length for each tissue specimen collection site in biopsy plan data 238.

At action 418, biopsy planning module 214 generates biopsy visualization interface 702. According to embodiments, biopsy planning module 214 may access a real-time tissue image or stored tissue images in images data 232, 3D tissue model data stored in 3D model data 234 and biopsy plan data 236, and may generate 2D or 3D biopsy visualization interface 702. Biopsy planning module 214 may store biopsy visualization interface 702 in biopsy plan data 238. Graphical user interface module 206 may access biopsy plan data 238, and may display one or more biopsy visualization interfaces 702 on display device 148.

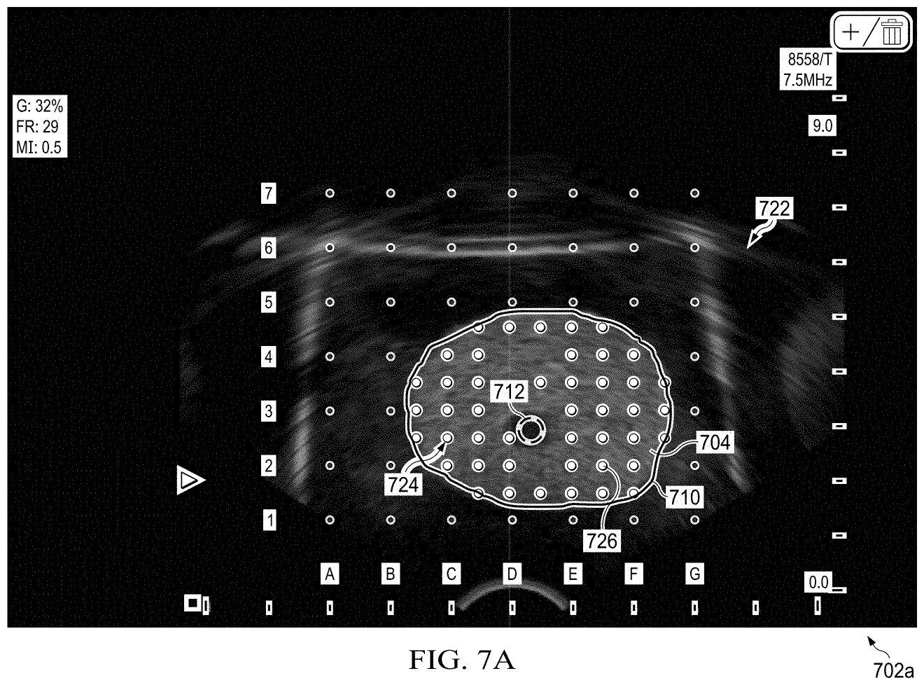

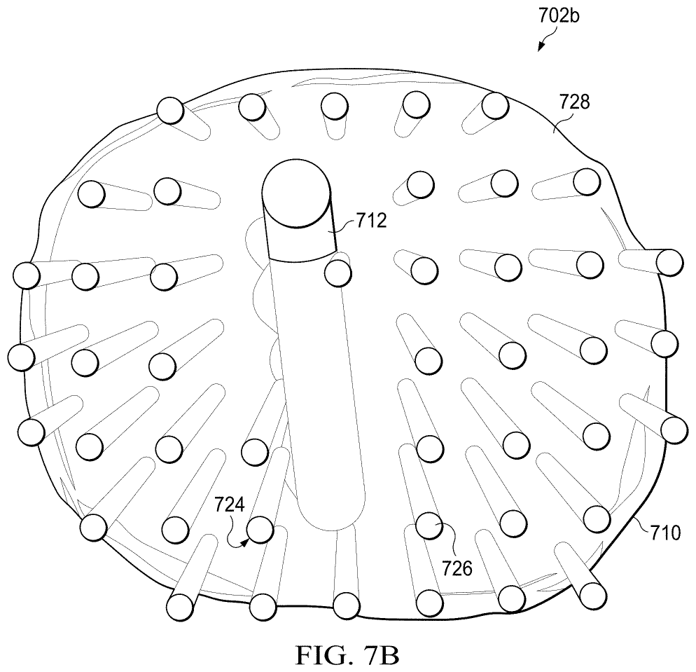

FIGS. 7A-7B illustrate exemplary biopsy visualization interfaces 702a and 702b, according to embodiments. According to embodiments, biopsy planning module 214 may generate biopsy visualization interfaces 702a and 702b to visualize the currently-selected tissue specimen collection sites on either a real-time or stored tissue image (best illustrated by FIG. 7A) or the 3D tissue model (best illustrated by FIG. 7B), and to visualize the effect of modifications to the biopsy plan as viewed against a real-time or stored tissue image or the 3D tissue model. By way of example only and not by way of limitation, modifications to the biopsy plan may alter the number of tissue specimens that will be collected during the biopsy, the size of the cannula and mandrel 114 used by needle assembly 110 to collect tissue specimens for biopsy and the according diameter of the tissue specimen collected, the minimum distance between tissue specimen collection sites, the specific location in alignment coordinates of one or more tissue specimen collection sites, the length of the tissue specimen that needle assembly 110 will collect at a particular tissue specimen collection site, and/or any other biopsy plan parameter.

FIG. 7A illustrates biopsy visualization interface 702a, according to an embodiment. Biopsy visualization interface 702a comprises real-time tissue image 722, tissue specimen collection grid 724, a plurality of tissue specimen collection sites 726, prostate tissue 704, prostate boundary contour 710, and urethra boundary contour 712. Although biopsy visualization interface 702a, real-time tissue image 722, tissue specimen collection grid 724, a plurality of tissue specimen collection sites 726, prostate tissue 704, prostate boundary contour 710, and urethra boundary contour 712 are shown and described in an exemplary configuration, embodiments contemplate any number or configuration of biopsy visualization interfaces 702a, real-time tissue images 722, tissue specimen collection grids 724, tissue specimen collection sites 726, prostate tissue 704, any other tissue, prostate boundary contours 710, and/or urethra boundary contours 712, according to particular needs.

In an embodiment, biopsy visualization interface 702a comprises tissue specimen collection grid 724 superimposed over real-time tissue image 722. In this embodiment, imaging module 204 controls imaging system 130 to generate real-time tissue image 722, and stores real-time tissue image 722 in images data 232. Graphical user interface module 206 accesses images data 232 and displays real-time tissue image 722 on display device 148. According to embodiments, biopsy planning module 214 accesses alignment data 236 and biopsy plan data 238, and superimposes tissue specimen collection grid 724, comprising a plurality of tissue specimen collection sites 726, over real-time tissue image 722 on display device 148 according to alignment coordinates stored in alignment coordinates stored in alignment data 236. Biopsy planning module 214 and graphical user interface module 206 may generate biopsy visualization interface 702a in the above manner to visualize the precise placement of tissue specimen collection grid 724 and the plurality of tissue specimen collection sites 726 on real-time tissue image 722, include the actual volume of tissue specimens that will be collected at each tissue specimen collection site 726 and the actual distance between tissue specimen collection sites 726 on real-time tissue image 722. In an embodiment, biopsy planning module 214 may switch between biopsy visualization interfaces 702a and 702b in response to input from input device 146.

FIG. 7B illustrates biopsy visualization interface 702b, according to an embodiment. Biopsy visualization interface 702b comprises 3D tissue model 728, tissue specimen collection grid 724, a plurality of tissue specimen collection sites 726, prostate boundary contour 710, and urethra boundary contour 712. Although biopsy visualization interface 702b, 3d tissue model 728, tissue specimen collection grid 724, a plurality of tissue specimen collection sites 726, prostate boundary contour 710, and urethra boundary contour 712 are shown and described in an exemplary configuration, embodiments contemplate any number or configuration of biopsy visualization interfaces 702b, 3d tissue models 728, tissue specimen collection grids 724, tissue specimen collection sites 726, prostate boundary contours 710, and/or urethra boundary contours 712, according to particular needs.

In an embodiment, biopsy visualization interface 702b comprises tissue specimen collection grid 724 viewed as a component of 3D tissue model 728. In this embodiment, biopsy planning module 214 stores tissue specimen collection grid 724 and a plurality of tissue specimen collection sites 726 in biopsy plan data 238, and stores tissue specimen collection grid 724 and a plurality of tissue specimen collection sites 726 as components of the 3D tissue model stored in 3D model data 234. Graphical user interface module 206 accesses 3D model data 234, and displays 3d tissue model 728 comprising tissue specimen collection grid 724 and a plurality of tissue specimen collection sites 726 on display device 148. Biopsy planning module 214 and graphical user interface module 206 may generate biopsy visualization interface 702b in the above manner to visualize the precise placement of tissue specimen collection grid 724 and the plurality of tissue specimen collection sites 726 on 3d tissue model 728, include the actual volume of tissue specimens that will be collected at each tissue specimen collection site 726 and the actual distance between tissue specimen collection sites 726 on 3d tissue model 728. In an embodiment, biopsy planning module 214 and graphical user interface module 206 may rotate 3d tissue model 728 in 3D space in response to input from input device 146.

Continuing the example, at action 418, biopsy planning module 214 and graphical user interface module 206 generate biopsy visualization interface 702b in the manner described above. In this example, biopsy visualization interface 702b displays the 3D prostate model, comprising tissue specimen collection grid 206 and a plurality of tissue specimen collection sites 726, on display device 148.

At action 420, and according to embodiments, biopsy planning module 214 analyzes, using the 3D tissue model stored in 3D model data 234 and the biopsy plan and tissue specimen collection grid stored in biopsy plan data 238, the volume and coordinates of the tissue that will be extracted for sampling according to the biopsy plan. Biopsy planning module 214 may determine whether one or more regions of the 3D tissue model are not adequately sampled by nearby tissue specimen collection sites, and may therefore harbor lesions, cancer, or other tissue abnormalities. Biopsy planning module may store the presence and location in 3D coordinates of one or more insufficiently-sampled regions (henceforth, “tissue gaps”) on the 3D tissue model in 3D model data 234. Biopsy planning module 214 calculates the presence and location in 3D coordinates of one or more tissue gaps on the 3D tissue model and biopsy plan, including but not limited to placing tissue gaps in any tissue region that is more than a specified distance from a tissue specimen collection site (such as, for example, any tissue region on the 3D tissue model that is more than 1 centimeter in any direction from any tissue specimen collection sites).

Continuing the example, at action 420, biopsy planning module 214 accesses the 3D prostate model stored in 3D model data 234 and the current biopsy plan and tissue specimen collection grid stored in biopsy plan data 238. In this example, biopsy planning module 214 determines that any location on the 3D prostate model that is not within at least 1 centimeter in any axis of a tissue specimen collection site qualifies as a tissue gap. In this example, biopsy planning module 214 locates a single tissue gap on the 3D prostate model based on the current biopsy plan. Biopsy planning module stores the location on the 3D prostate model of the tissue gap in 3D model data 234.

At action 422, and according to embodiments, biopsy planning module 214 displays the one or more tissue gaps located at action 420 on biopsy visualization interfaces 702a. As described in greater detail below, biopsy planning module 214 displays one or more tissue gaps to highlight potential problems or deficiencies with the current biopsy plan, and to provide the opportunity (at action 424) to adjust the biopsy plan to accommodate these deficiencies, such as, for example, by adding a new tissue specimen collection site to the biopsy plan to sample tissue in the tissue gap. Although biopsy planning module 214 is described herein as displaying one or more tissue gaps on biopsy visualization interface 702a, in other embodiments biopsy planning module may display one or more tissue gaps on biopsy visualization interface 702b.

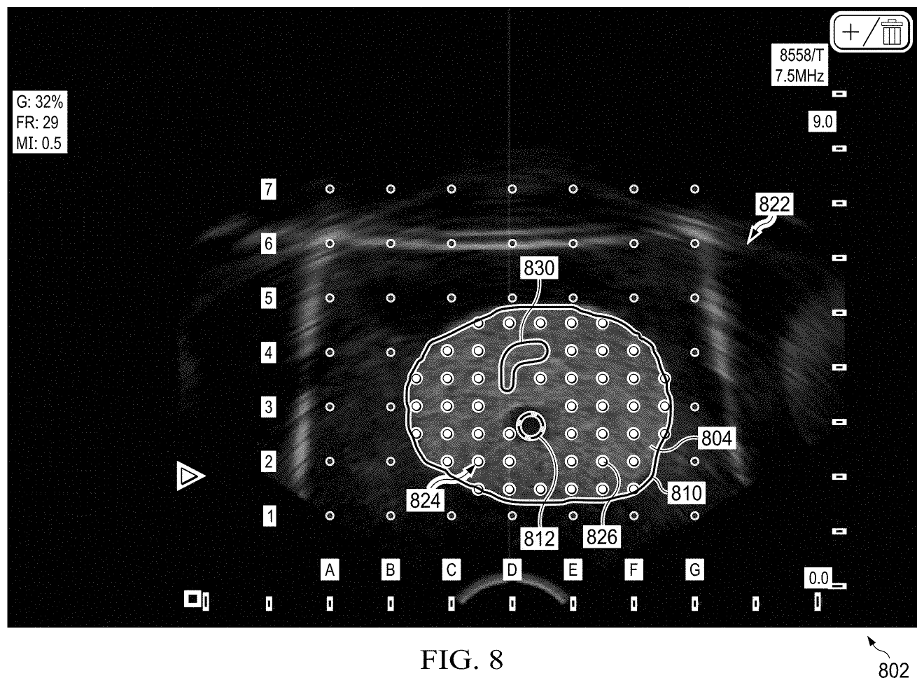

FIG. 8 illustrates biopsy planning module 214 displaying tissue gap 830 on biopsy visualization interface 802, according to an embodiment. In this embodiment, biopsy visualization interface 802 comprises real-time tissue image 822, tissue specimen collection grid 824, a plurality of tissue specimen collection sites 826, prostate tissue 804, prostate boundary contour 810, urethra boundary contour 812, and tissue gap 830. Although biopsy visualization interface 802, real-time tissue image 822, tissue specimen collection grid 824, a plurality of tissue specimen collection sites 826, prostate tissue 804, prostate boundary contour 810, urethra boundary contour 812, and tissue gap 830 are shown and described in an exemplary configuration, embodiments contemplate any number or configuration of biopsy visualization interfaces 802, real-time tissue images 822, tissue specimen collection grids 824, tissue specimen collection sites 826, prostate tissue 804, any other tissue, prostate boundary contours 810, urethra boundary contours 812, and/or tissue gaps 830, according to particular needs.

As described above, imaging module 204 may control imaging system 130 to generate real-time tissue image 822, and stores real-time tissue image 822 in images data 232. Graphical user interface module 206 accesses images data 232 and displays real-time tissue image 822 on display device 148. Biopsy planning module 214 accesses alignment data 236 and biopsy plan data 238, and superimposes tissue specimen collection grid 824, comprising a plurality of tissue specimen collection sites 826, and tissue gap 830 over real-time tissue image 822 on display device 148 according to alignment coordinates stored in alignment coordinates stored in alignment data 236. Tissue gap 830 indicates a tissue region of the real-time tissue image 822 that the current biopsy plan and tissue specimen collection grid 824 does not sample.

Continuing the example, at action 422, biopsy planning module 214, imaging module 204, and graphical user interface module 206 generate biopsy visualization interface 802, containing tissue gap 830, in the manner described above.

At action 424, biopsy planning module 214 determines whether to modify the biopsy plan to re-configure the number of tissue specimens that will be collected during the biopsy, the size of the cannula and mandrel 114 used by needle assembly 110 to collect tissue specimens for biopsy and the according diameter of the tissue specimen collected, the minimum distance between tissue specimen collection sites, the specific location in alignment coordinates of one or more tissue specimen collection sites, the length of the tissue specimen that needle assembly 110 will collect at a particular tissue specimen collection site, and/or any other biopsy plan parameter. If biopsy planning module 214 chooses not to modify the biopsy plan, biopsy planning module 214 terminates method 400. If biopsy planning module 214 does choose to modify the biopsy plan, biopsy planning module 214 moves to action 426, modifies the biopsy plan, and moves to action 422 to continue executing method 400.

By way of example only and not by way of limitation, biopsy planning module 214 may modify the biopsy plan at action 426 in response to one or more tissue gaps 830, to change needle gauge size of needle assembly 110 (thereby requiring the recalculation of tissue specimen collection sites on the tissue specimen collection grid), or for any other reason. In an embodiment, biopsy planning module 214 and graphical user interface module 206 continuously generate updated biopsy visualization interfaces 702a and 702b in the manner described above to visualize modification made to the biopsy plan. In an embodiment, biopsy planning module 214 modifies the biopsy plan in response to input received by input device 146.

In an embodiment, biopsy planning module 214 may modify the biopsy plan at action 426 by inserting an additional tissue specimen collection site. In this embodiment, biopsy planning module 214 and graphical user interface module 206 may generate and overlay a graphical user interface cursor, in the manner described above, over biopsy visualization interface 702a or 702b. Biopsy planning module 214 may move the graphical user interface cursor on biopsy visualization interface 702a or 702b in response to input received from input device 146. In an embodiment, a medical professional may operate input device 146 to (1) move the graphical user interface cursor over a location on the real-time tissue image or 3D tissue model and generate an additional tissue specimen collection site at that location; (2) move the graphical user interface cursor over an extant tissue specimen collection site and delete the tissue specimen collection site; and/or (3) move the graphical user interface cursor over an extant tissue specimen collection site, select the extant tissue specimen collection site, and make any modifications to the extant tissue specimen collection site. Such modification my include, for example, configuring the size of the cannula and mandrel 114 used by needle assembly 110 to collect a tissue specimen for biopsy and the according diameter of the tissue specimen collected at the tissue specimen collection site, the length of the tissue specimen that needle assembly 110 will collect at the tissue specimen collection site, and/or any other tissue specimen collection site parameter. In an embodiment, biopsy planning module 214 and graphical user interface module 206 continuously generate updated biopsy visualization interfaces 702a and 702b in the manner described above to visualize modification made to the biopsy plan.

Concluding the example, biopsy planning module 214 at action 424 chooses to modify the biopsy plan. At action 426, biopsy planning module 214 modifies the biopsy plan by adding an additional tissue specimen collection site in the middle of tissue gap 830. Biopsy planning module 214 stores the updated biopsy plan in biopsy plan data 238, moves to action 422 and displays tissue gaps in the manner described above, chooses not to modify the biopsy plan further at action 424, and method 400 ends.

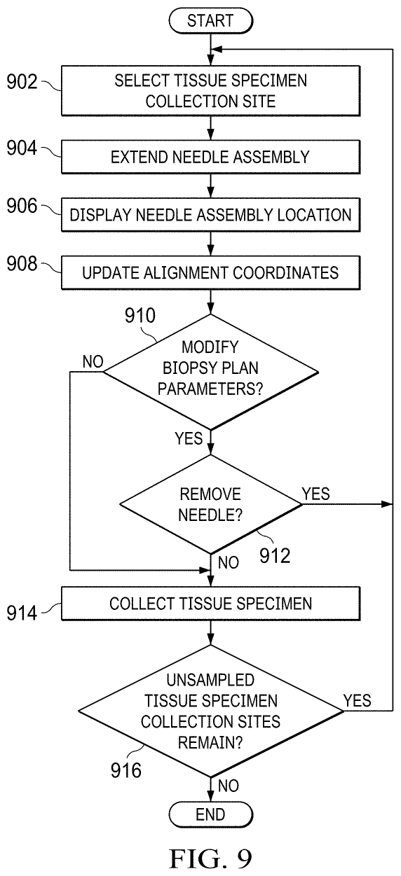

FIG. 9 illustrates exemplary method 900 of conducting a biopsy phase, according to an embodiment. In an embodiment, 3D mapping system 140 performs the actions of method 900 to collect tissue specimens from patient 160 according to a biopsy plan. Although actions of method 900 are described in a particular order, embodiments contemplate actions performed in any suitable order or combination according to particular needs.

At action 902, and according to embodiments, tissue collection module 216 accesses a biopsy plan stored in biopsy plan data 238 and selects a tissue specimen collection site. In an embodiment, prior to action 902, 3D mapping system 140 executing the actions of method 400 generates, modifies, and stores a biopsy plan in biopsy plan data 238. In other embodiments, 3D mapping system 140 may receive and store in biopsy plan data 238 a pre-generated biopsy plan.

According to embodiments, at action 902, tissue collection module 216 selects a tissue specimen collection site on the biopsy plan from which tissue specimen collection module 216 has not yet collected a tissue specimen sample (henceforth, the “unsampled tissue specimen collection site”). According to embodiments, tissue collection module 216 may select the unsampled tissue specimen collection site that is nearest to the current position of actuator assembly 120 and needle assembly 110. In other embodiments, tissue collection module 216 may begin with the first-numbered tissue specimen collection site, proceed to the next-numbered tissue specimen collection site after collecting a tissue specimen at the first tissue specimen collection site, and so on. In an embodiment, tissue collection module 216 automatically numbers the subsequent biopsy site in numerical order, and in an embodiment in which 3D mapping system 140 adds a new tissue specimen collection site to the biopsy plan because of a tissue gap or for any other reason, tissue collection module 216 automatically numbers the new site to follow the preceding biopsy site and renumbers subsequent tissue specimen collection sites accordingly, further resulting in all unsampled tissue specimen collection sites to increase their site number. Having selected an unsampled tissue specimen collection site, tissue collection module 216 accesses alignment data 236, and moves actuator assembly 120 to the alignment coordinates of the unsampled tissue specimen collection site.

To provide an example of 3D mapping system 140 executing the actions of method 900 and not by way of limitation, 3D mapping system 140 at action 902 prepares to collect biopsy tissue specimens from the prostate of patient 160. Tissue collection module 216 accesses a prostate biopsy plan stored in biopsy plan data 238 and a 3D prostate model stored in 3D model data 234. As best illustrated by FIG. 10 below, tissue collection module 216 selects unsampled tissue specimen collection site 1032 from which to collect a tissue specimen. Tissue collection module 216 accesses alignment data 236, and moves actuator assembly 120 to the alignment coordinates of unsampled tissue specimen collection site 1032.

At action 904, and according to embodiments, tissue collection module 216 accesses biopsy plan data 238 and actuates actuator assembly 120 to extend needle assembly 110 into patient 160 at the unsampled tissue specimen collection site specified at action 902. Tissue collection module 216 may operate actuator assembly 120 to extend needle assembly 110 a controlled distance into patient 160 (such as, for example, 4.1 centimeters) based on (1) the unsampled tissue specimen collection site's distance from actuator assembly 120; (2) intervening tissue separating the unsampled tissue specimen collection site from actuator assembly 120; or (3) any other tissue specimen collection site information stored in biopsy plan data 238.

Continuing the example, at action 904, tissue collection module 216 accesses biopsy plan data 238, and actuates actuator assembly 120 to extend needle assembly 110 into patient 160 at the location of unsampled tissue specimen collection site 1032. In this example, the biopsy plan information for unsampled tissue specimen collection site 1032 specifies extending needle assembly 110 6.4 centimeters into patient 160 to reach unsampled tissue specimen collection site 1032, and collecting a tissue specimen 2.1 centimeters in length once needle assembly 110 is in place. Tissue collection module 216 actuates actuator assembly 120 to extend needle assembly 110 6.4 centimeters into patient 160 at unsampled tissue specimen collection site 1032. Although particular examples of needle assembly 110 extension lengths and tissue specimen sample lengths are described herein, embodiments contemplate any needle assembly 110 extension lengths and/or tissue specimen sample lengths, according to particular needs.

At action 906, tissue collection module 216 displays the location of needle assembly 110 on display device 148. Actions 906 enables tissue collection module 216 to determine whether needle assembly 110 was in fact extended into the correct unsampled tissue specimen collection site alignment coordinates at action 904. In an embodiment, at action 906, imaging module 204 controls imaging system 130 to generate a real-time tissue image, including the current location of needle assembly 110 extended into the tissue, and stores the real-time tissue image in images data 232. Graphical user interface module 206 accesses images data 232 and displays the real-time tissue image, including the current location of needle assembly 110, on display device 148. Tissue collection module 216 accesses the 3D tissue model stored in 3D model data 234 and the biopsy plan stored in biopsy plan data 238, and superimposes the biopsy plan over the real-time tissue image on display device 148.

FIG. 10 illustrates exemplary tissue specimen collection visualization interface 1002, according to an embodiment. According to embodiments, tissue collection module 216 may generate tissue specimen collection visualization interface 1002 to display the in-tissue location and alignment coordinates of needle assembly 110 as compared to the intended tissue specimen collection site according to the biopsy plan. Tissue specimen collection visualization interface 1002 may comprise real-time tissue image 1022, tissue specimen collection grid 1024, a plurality of tissue specimen collection sites 1026, unsampled tissue specimen collection site 1032, prostate tissue 1004, prostate boundary contour 1010, urethra boundary contour 1012, and needle assembly 110. Although tissue specimen collection visualization interface 1002, real-time tissue image 1022, tissue specimen collection grid 1024, plurality of tissue specimen collection sites 1026, unsampled tissue specimen collection site 1032, prostate tissue 1004, prostate boundary contour 1010, urethra boundary contour 1012, and needle assembly 110 are shown and described in an exemplary configuration, embodiments contemplate any number or configuration of tissue specimen collection visualization interfaces 1002, real-time tissue images 1022, tissue specimen collection grids 1024, tissue specimen collection sites 1026, prostate tissue 1004, any other tissue, prostate boundary contours 1010, urethra boundary contours 1012, or needles 1010, according to particular needs.

In an embodiment, tissue specimen collection visualization interface 1002 comprises tissue specimen collection grid 1024 superimposed over real-time tissue image 1022. In this embodiment, imaging module 204 controls imaging system 130 to generate real-time tissue image 1022, which includes needle assembly 110 viewed from an end-on perspective, and stores real-time tissue image 1022 in images data 232. Graphical user interface module 206 accesses images data 232 and displays real-time tissue image 1022 and needle assembly 110 on display device 148. According to embodiments, tissue collection module 216 accesses alignment data 236 and biopsy plan data 238, and superimposes tissue specimen collection grid 724, comprising a plurality of tissue specimen collection sites 726 including unsampled tissue specimen collection site 1032, over real-time tissue image 722 on display device 148 according to alignment coordinates stored in alignment coordinates stored in alignment data 236. Tissue collection module 216 and graphical user interface module 206 may generate tissue specimen collection visualization interface 1002 in the above manner to visualize the distance between needle assembly 110 and unsampled tissue specimen collection site 1032, and to determine whether tissue collection module 216 extended needle assembly 110 into the correct location during action 904.

Continuing the example, at action 906, tissue collection module 216 and graphical user interface module 206 generate tissue specimen collection visualization interface 1002 in the manner described above. In this example, tissue specimen collection visualization interface 1002 displays real-time prostate tissue image 1022, including the real-time location of needle assembly 110 viewed from an end-on perspective. Tissue specimen collection visualization interface 1002 superimposes tissue specimen collection grid 1024, including unsampled tissue specimen collection site 1032, over real-time prostate tissue image 1022. In this example, and as illustrated by tissue specimen collection visualization interface 1002, tissue collection module 216 extended needle assembly 110 into a region of prostate tissue directly below the intended unsampled tissue specimen collection site 1032.

At action 908, tissue collection module 216 updates the alignment coordinates of the unsampled tissue specimen collection site, stored in the biopsy plan data 238, to reflect the alignment coordinates of needle assembly 110 displayed on tissue specimen collection visualization interface 1002. As described in greater detail below, action 908 permits tissue collection module 216 to update the biopsy plan and tissue specimen collection grid with the actual location, measured in alignment coordinates, into which tissue collection module 216 extended needle assembly 110 at action 904.

In an embodiment, tissue collection module 216 and graphical user interface module 206 may generate and overlay a graphical user interface cursor, in the manner described above, over tissue specimen collection visualization interface 1002. Tissue collection module 216 may move the graphical user interface cursor on tissue specimen collection visualization interface 1002 in response to input received from input device 146. Tissue collection module 216 may use the graphical user interface cursor on tissue specimen collection visualization interface 1002 to update the alignment coordinates of the unsampled tissue specimen collection site, stored in the biopsy plan data 238, to reflect the alignment coordinates of needle assembly 110 displayed on tissue specimen collection visualization interface 1002. By way of example only and not by way of limitation, in an embodiment, a medical professional may operate input device 146 (in this example, comprising a computer mouse) to (1) move the graphical user interface cursor over the unsampled tissue specimen collection site, (2) click and hold the mouse button to select the unsampled tissue specimen collection site, (3) drag the unsampled tissue specimen collection site over the location of needle assembly 110 on tissue specimen collection visualization interface 1002, and (4) release the mouse button to update the alignment coordinates of the unsampled tissue specimen collection site to reflect the alignment coordinates of needle assembly 110. Tissue collection module 216 may store the updated alignment coordinates of the unsampled tissue specimen collection site in alignment data 236.

Continuing the example, at action 908, tissue collection module 216 and graphical user interface module 206 generate and overlay a graphical user interface cursor over tissue specimen collection visualization interface 1002. In this example, a medical professional operates a computer mouse to (1) move the graphical user interface cursor over unsampled tissue specimen collection site 1032, (2) click and hold the mouse button to select unsampled tissue specimen collection site 1032, (3) drag unsampled tissue specimen collection site 1032 downward to superimpose over needle assembly 110 on tissue specimen collection visualization interface 1002, and (4) release the mouse button to update the alignment coordinates of unsampled tissue specimen collection site 1032 to reflect the alignment coordinates of needle assembly 110. Tissue collection module 216 stores the updated alignment coordinates of the unsampled tissue specimen collection site in alignment data 236.

At action 910, and according to embodiments, tissue collection module 216 accesses alignment data 236, biopsy plan data 238, and stop criteria 246, and determines whether to modify one or more biopsy plan parameters for the unsampled tissue specimen collection site. Action 910 enables tissue collection module 216 to adapt the biopsy plan to reflect unintended changes introduced by the extension of needle assembly 110 into tissue alignment coordinates that do not precisely align with the alignment coordinates the biopsy plan specifies for the unsampled tissue specimen collection site.

In an embodiment, stop criteria 246 may instruct tissue collection module 216 to reconfigure the remaining biopsy plan tissue specimen collection sites (such as, for example, by adding additional tissue specimen collection sites to the biopsy plan) if tissue collection module 216 extended needle assembly 110 into a tissue region that deviated from the alignment coordinates specified by the biopsy plan by more than a specified distance (such as, for example, 0.5 centimeters). In other embodiments, stop criteria 246 may instruct tissue collection module 216 to alter the length of the tissue specimen that needle assembly 110 will collect at the current location of needle assembly 110 (such as, for example, by reducing the originally-intended tissue specimen collection length to avoid puncturing the prostate tissue boundary based on the current position of needle assembly 110). Other embodiments may comprise stop criteria 246 instructing tissue collection module 216 to move to action 912 and determine whether to retract needle assembly 110 from patient 160, as described in greater detail below, if tissue collection module 216 extended needle assembly 110 into a tissue region that deviated from the alignment coordinates specified by the biopsy plan by more than a specified distance (such as, for example, 2.0 centimeters). The above examples of stop criteria 246 are provided for exemplary purposes only, and embodiments contemplate any form of stop criteria 246 altering the operation of tissue collection module 216 in response to tissue collection module 216 extending needle assembly 110 into patient 160 at a tissue location not specified by the biopsy plan. If tissue collection module 216 determines no stop criteria 246 have occurred—such as, for example, in an embodiment in which tissue collection module 216 extended needle assembly 110 into the correct region of patient tissue 216 specified by the biopsy plan—tissue collection module 216 moves to action 914.

At action 912, tissue collection module 216 accesses stop criteria 246 and determines whether to retract needle assembly 110 from patient 160. If tissue collection module 216 extended needle assembly 110 into a tissue region that deviated from the alignment coordinates specified by the biopsy plan by more than a specified distance (such as, for example, 2.0 centimeters) according to stop criteria 246, tissue collection module 216 actuates actuator assembly 120 to retract needle assembly 110 from patient 160. Having retracted needle assembly 110, tissue collection module 216 moves to action 902 and continues the actions of method 900 described above. In an embodiment in which tissue collection module 216 accesses stop criteria 246 and determines tissue collection module 216 did not extend needle assembly 110 into a tissue region that so deviated from the specified alignment coordinates as to require needle assembly 110 retraction, tissue collection module 216 may move to action 914.

Continuing the example, at action 910, tissue collection module 216 accesses alignment data 236, biopsy plan data 238, and stop criteria 246, and determines that although tissue collection module 216 did not extend needle assembly 110 precisely into unsampled tissue specimen collection site 1032 as specified by the biopsy plan, the deviation introduced was not sufficient to trigger any stop criteria 246. Tissue collection module continues to action 914.

At action 914, and according to embodiments, tissue collection module 216 collects a tissue specimen. In an embodiment, tissue collection module 216 actuates actuator assembly 120 to move needle assembly 110 cannula with respect to needle assembly 110 mandrel 114, thereby exposing one or more needle assembly 110 core bed 116 protrusions. Tissue collection module 216 further actuates actuator assembly 120 to impress upon one or more core bed 116 protrusions a tissue specimen on core bed 116 projections of needle assembly 110. Tissue collection module 216 actuates actuator assembly 120 to move needle assembly 110 cannula with respect to needle assembly 110 mandrel 114 to cover one or more needle assembly 110 core bed 116 protrusions. Having secured a tissue specimen, tissue collection module 216 actuates actuator assembly 120 to retract needle assembly 110 from patient 160. Tissue collection module 216 stores the alignment coordinates from which needle 110 collected the tissue specimen in tissue collection data 240. Tissue collection module 216 stores the tissue specimen in actuator assembly 120 for subsequent biopsy and analysis.

Continuing the example, at action 910, tissue collection module 216 collects a tissue specimen from unsampled tissue specimen collection site 1032 in the manner described above. Tissue collection module retracts needle assembly 110 from the now-sampled tissue specimen collection site 1032, and stores the alignment coordinates from which needle 110 collected the tissue specimen in tissue collection data 240.

At action 916, tissue collection module 216 accesses the biopsy plan stored in biopsy plan data 238, and determines whether additional unsampled tissue specimen collection sites remain in the biopsy plan. If additional unsampled tissue specimen collection sites remain, tissue collection module 216 returns to action 902 and continues performing the actions of method 900. If tissue collection module 216 has collected samples from all tissue specimen collection sites specified by the biopsy plan, tissue collection module 216 terminates method 900. Concluding the example, tissue collection module 216 at action 916 determines additional unsampled tissue specimen collection sites remain in the prostate biopsy plan. Tissue collection module 216 returns to action 902, and executes the actions method 900 until tissue collection module 216 collects tissue specimens from all tissue specimen collection sites specified by the biopsy plan.

In an embodiment, at any action of method 900, tissue collection module 216 may access biopsy plan data 238 and make modifications to the biopsy plan. By way of example only and not by way of limitation, such modifications to the biopsy plan may include re-configuring the number of tissue specimen collection sites into which tissue collection module 216 will extend needle assembly 110 according to the biopsy plan; the size of the cannula and mandrel 114 used by needle assembly 110 to collect tissue specimens for biopsy and the according diameter of the tissue specimen collected; the minimum distance between tissue specimen collection sites; the specific location in alignment coordinates of one or more tissue specimen collection sites; the length of the tissue specimen that needle assembly 110 will collect at a particular tissue specimen collection site; and/or any other biopsy plan parameter. In an embodiment, tissue collection module 216 modifies the biopsy plan in response to input received by input device 146. In other embodiments, biopsy planning module 214 may recalculate the presence of one or more tissue gaps, as described above, based one or more modifications made to the biopsy plan during method 900. If biopsy planning module 214 determines that modifications to the biopsy plan have created one or more new tissue gaps, biopsy planning module 214 may display the one or more new tissue gaps on tissue specimen collection visualization interface 1002, in the manner described above with respect to biopsy visualization interface 702.

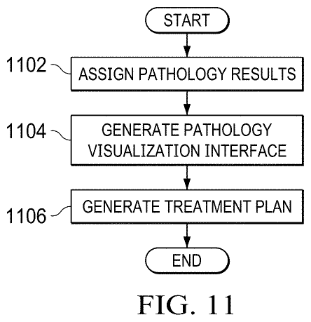

FIG. 11 illustrates exemplary method 1100 of conducting a pathology phase, according to an embodiment. In an embodiment, 3D mapping system 140 performs the actions of method 1100 to review biopsy results and develop a treatment plan to respond to one or more lesions, cancer, or other tissue anomalies located in the biopsy results. Although actions of method 1100 are described in a particular order, embodiments contemplate actions performed in any suitable order or combination according to particular needs.

At action 1102, and according to embodiments, pathology module 218 assigns pathology results to the tissue specimens collected by 3D mapping system 140 executing the actions in method 900. In an embodiment, the tissue specimens previously collected by tissue collection module 216 are biopsied. Server 142 stores the pathology results of each tissue specimen biopsy in pathology data 242. Server 142 may store any form of pathology information regarding each tissue specimen in pathology data 242, including but not limited to the length of the tissue sample occupied by lesions, cancer, or any other tissue anomalies as compared to the overall tissue sample; the alignment coordinates of the tissue sample lesions, cancer, or any other tissue anomalies (as measured in millimeters, 3D tissue model coordinates, or any other unit of measurement); tissue Gleason score assigning a numerical value to the aggressiveness of cancer observed in the tissue specimen; any pathology remarks entered by the medical professional, or any other form of pathology information. In an embodiment, at action 1102, pathology module 218 accesses pathology results for each tissue specimen stored in pathology data 242, and stores the pathology results for each tissue specimen in tissue collection data 240.

At action 1104, pathology module 218 generates pathology visualization interface 1202. According to embodiments, pathology module 218 may access a real-time tissue image or stored tissue images in images data 232, 3D tissue model data stored in 3D model data 234, and tissue specimen data stored in tissue collection data 240, and may generate 2D or 3D pathology visualization interface 1202. Pathology module 218 may store pathology visualization interface 1202 in pathology data 242. Graphical user interface module 206 may access pathology data 242, and may display one or more pathology visualization interfaces on display device 148.

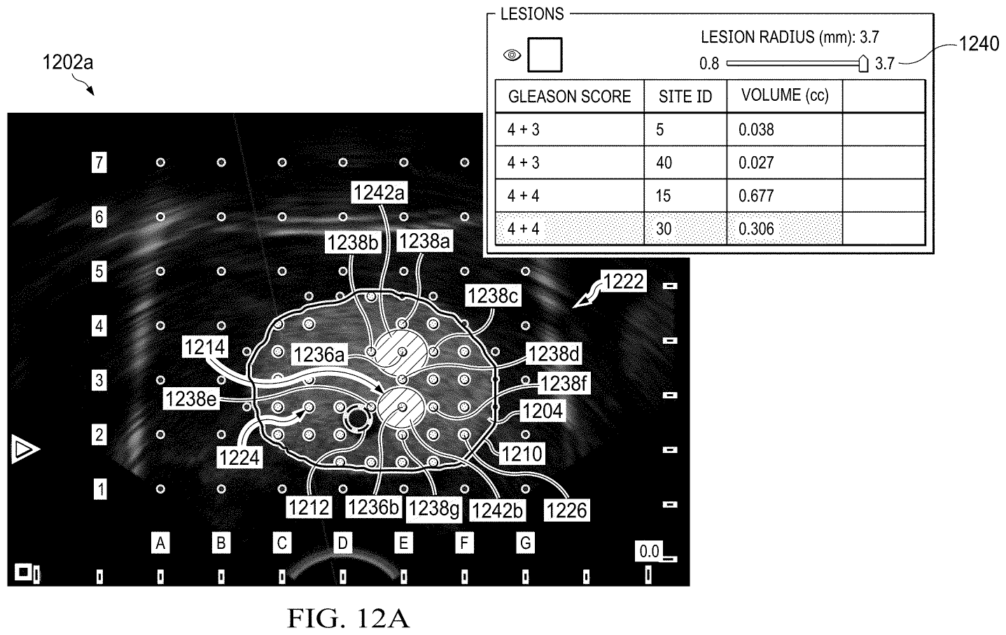

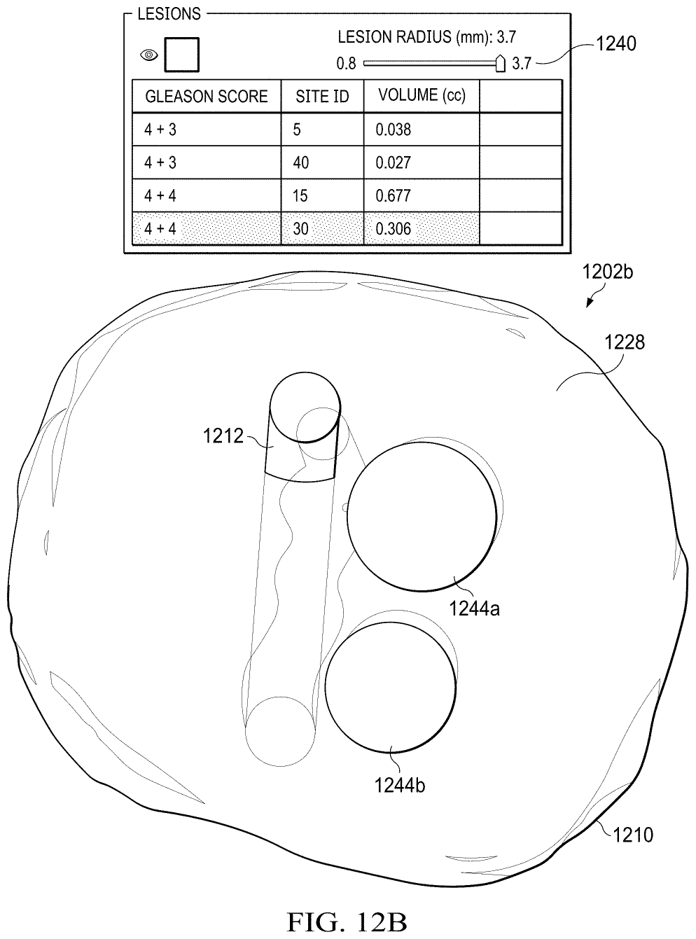

FIGS. 12A-12B illustrate exemplary pathology visualization interfaces 1202a and 1202b, according to embodiments. According to embodiments, pathology module 218 may generate pathology visualization interfaces 1202a and 1202b to visualize the tissue specimen pathology results on either a real-time or stored tissue image (best illustrated by FIG. 12A) or the 3D tissue model (best illustrated by FIG. 12B), and to visualize one or more potential treatment options.

FIG. 12A illustrates pathology visualization interface 1202a, according to an embodiment. Pathology visualization interface 1202a comprises tissue image 1222, tissue specimen collection grid 1224, a plurality of tissue specimen collection sites 1226, lesion sites 1236a and 1236b, lesion-neighboring sites 1238a-1238g, lesion slider 1240, lesion radius 1242a and 1242b, prostate tissue 1204, prostate boundary contour 1210, and urethra boundary contour 1212. Although pathology visualization interface 1202a, tissue image 1222, tissue specimen collection grid 1224, a plurality of tissue specimen collection sites 1226, lesion sites 1236a and 1236b, lesion-neighboring sites 1238a-1238g, lesion slider 1240, lesion radius 1242a and 1242b, prostate tissue 1204, prostate boundary contour 1210, and urethra boundary contour 1212 are shown and described in an exemplary configuration, embodiments contemplate any number or configuration of pathology visualization interfaces 1202a, real-time tissue images 1222, tissue specimen collection grids 1224, tissue specimen collection sites 1226, lesion sites 1236a-1236b, lesion-neighboring sites 1238a-1238g, lesion sliders 1240, lesion radii 1214, prostate tissue 1204, any other tissue, prostate boundary contours 1210, and/or urethra boundary contours 1212, according to particular needs.

In an embodiment, pathology visualization interface 1202a comprises tissue specimen collection grid 1224 superimposed over tissue image 1222. In this embodiment, imaging module 204 accesses tissue image 1222 stored in images data 232. Graphical user interface module 206 accesses images data 232 and displays tissue image 1222 on display device 148. According to embodiments, pathology module 218 accesses alignment data 236, biopsy plan data 238, and tissue specimen collection data 240. Pathology module 218 superimposes tissue specimen collection grid 1224, comprising a plurality of tissue specimen collection sites 1226, over tissue image 1222 on display device 148 according to alignment coordinates stored in alignment data 236. Pathology module 218 uses tissue specimen collection data 240 to indicate tissue specimen collection sites 1226 that contain lesions, cancer, or other tissue abnormalities (in this illustrated example, lesion sites 1236a and 1236b) on pathology visualization interface 1202a.

In an embodiment, lesion sites 1236a and 1236b indicate tissue specimen collection sites 1226 that, when biopsied, were determined to contain lesions, cancer, or other tissue anomalies. In an embodiment not illustrated in FIG. 12A, pathology visualization interface 702 may indicate the alignment coordinates of boundaries between lesions and non-lesion tissue along the length of the tissue specimen collection sites 1226. Tissue specimen collection sites 1226 that do not display lesions indicate that no lesions were located during the biopsies of those tissue specimen collection sites 1226. Pathology module 218 may access tissue collection data 240 for each tissue specimen collection site 1226, and may highlight, using various colors or symbols, lesion sites 1226 of varying degrees of pathological severity, based on, for example, tissue Gleason scores, lesion sizes, pathology notes, and/or any other data stored in the pathology results for each tissue specimen stored in tissue collection data 240 and/or pathology data 242.

According to embodiments, pathology module 218 generates lesion slider 1240, and displays lesion slider 1240 on pathology visualization interface 1202a. Pathology module 218 may adjust lesion slider 1240 from left to right and vice-versa on pathology visualization interface 1202a in response to input received from input device 146. Pathology module 218 may control the sizes of lesion radius 1242a and 1242b in response to changes made to lesion slider 1240. In this manner, lesion slider 1240 may control the sizes of lesion radius 1242a and 1242b on pathology visualization interface 1202a.

Lesion slider 1240 and lesion radius 1242a and 1242b visualize on pathology visualization interface 1202a potential tissue regions of patient 160 to treat at action 308 of method 300. By way of example only and not by way of limitation, pathology module 218 may increase or decrease one or more lesion radii 1214, and thus the tissue area of patient 160 to be treated at action 308 of method 300, based on the severity of lesions, cancer, or other tissue anomalies detected at a particular lesion site 1236a or 1236b. Pathology module 218 may store the alignment coordinates of the one or more lesion radii 1214 in alignment data 236.

In an embodiment in which pathology module 218 assigns a high tissue Gleason score or other characterization to lesion site 1236a, pathology module 218 expands lesion radius 1242a to encompass lesion-neighboring sites 1238a-1238g. In this embodiment, lesion-neighboring sites 1238a-1238g tested negative for lesions at action 1102. By expanding lesion radius 1242a to encompass lesion-neighboring sites 1238a-1238g, and treating at action 308 the tissue encompassed within lesion radius 1242a (including the tissue located at lesion-neighboring sites 1238a-1238g), 3D mapping system 140 may ensure that lesion site 1236a is completely treated or removed.

In an embodiment, pathology module 218 may switch between pathology visualization interfaces 1202a and 1202b in response to input from input device 146. FIG. 12B illustrates pathology visualization interface 1202b, according to an embodiment. Pathology visualization interface 1202b comprises 3D tissue model 1228, lesion slider 1240, 3D lesion cylinders 1244a and 1244b, prostate boundary contour 1210, and urethra boundary contour 1212. Although pathology visualization interface 1202b, 3D tissue model 1228, lesion slider 1240, 3D lesion cylinders 1244a and 1244b, prostate boundary contour 1210, and urethra boundary contour 1212 are shown and described in an exemplary configuration, embodiments contemplate any number or configuration of pathology visualization interfaces 1202b, 3D tissue models 1228, lesion sliders 1240, 3D lesion cylinders 1244, prostate boundary contours 1210, and/or urethra boundary contours 1212, according to particular needs.

In an embodiment, pathology visualization interface 1202b comprises 3D lesion cylinders 1244a and 1244b viewed as components of 3D tissue model 1228. In this embodiment, pathology module 218 accesses tissue collection data 240, alignment data 236, and pathology data 242, and stores the location of one or more tissue specimen collection sites 1226 that contain lesions, cancer, or other tissue anomalies as 3D lesion cylinders 1244a and 1244b on 3D tissue model 1228 stored in 3D model data 234. Graphical user interface module 206 accesses 3D model data 234, and displays 3D tissue model 1228 comprising 3D lesion cylinders 1244a and 1244b on display device 148. Pathology module 218 and graphical user interface module 206 may generate pathology visualization interface 1202b in the above manner to visualize the location of one or more lesions, represented by 3D lesion cylinders 1244a and 1244b, on 3D tissue model 1228. In an embodiment, pathology module 218 and graphical user interface module 206 may rotate 3D tissue model 1228 in 3D space in response to input from input device 146.

In this embodiment, lesion slider 1240 operates in the manner described above with respect to pathology visualization interface 1202a. Pathology module 218 may control the sizes of 3D lesion cylinders 1244a and 1244b in response to changes made to lesion slider 1240. In this manner, lesion slider 1240 may control the sizes of 3D lesion cylinders 1244a and 1244b on pathology visualization interface 1202b. As described above, pathology module 218 may increase or decrease the size of one or more 3d lesion cylinders 1244, and thus the area of tissue to be treated at action 308 of method 300, based on the severity of lesions, cancer, or other tissue anomalies detected at a particular 3D lesion cylinders 1244a or 1244b. Pathology module 218 may store the alignment coordinates of the one or more 3D lesion cylinders 1244 in alignment data 236.

At action 1106, pathology module 218 generates a treatment plan. In an embodiment, pathology module 218 adjusts lesion slider 1240 on pathology visualization interfaces 1202a and/or 1202b in the manner described above to choose regions of tissue to treat during action 308 of method 300. In addition, lesion slider 1240 indicates lesion size and volume where this information can be used when deciding on appropriate treatment. A practitioner may use this information to treat the entire gland or part of it and may further decide on the type of lesion treatment based on values generated by lesion slider 1240. Having selected regions of tissue to treat, pathology module 218 generates a treatment plan, stores the treatment plan in treatment data 244, and terminates method 1100.

According to embodiments, the treatment plan may comprise any method to treat, irradiate, remove, or otherwise neutralize lesion sites 1236a located in the tissue of patient 160, including, in an embodiment, the tissue surrounding lesion sites 1236a. By way of example only and not by way of limitation, the treatment plan may comprise excising lesion sites 1236a and surrounding tissue surgically, implanting one or more radioactive pellets to irradiate lesion sites 1236a and surrounding tissue, or any other method to remove or neutralize lesion sites 1236a and surrounding tissue, including but not limited to injecting liquid, semi-liquid or solid materials into the lesions by utilizing the treatment plan. In an embodiment in which tissue specimen pathology results indicate a higher probability that a tumor or other lesion may have escaped the prostate gland or has the potential to do so, based on analysis of the tissue removed or otherwise, the treatment of the lesion may also be accompanied by a generalized or systemic treatment plan to damage, kill or destroy cancer or tumor cells that may be outside of the prostate gland or other organ previously biopsied by system 100. In an embodiment, pathology module 218 may store the alignment coordinates of the one or more tissue regions to treat according to the treatment plan in alignment data 236.

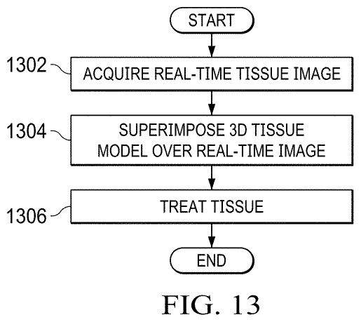

FIG. 13 illustrates exemplary method 1300 of conducting a treatment phase, according to an embodiment. In an embodiment, 3D mapping system 140 performs the actions of method 1300 to treat one or more tissue lesions, cancer, or other tissue anomalies according to a treatment plan. Although actions of method 1300 are described in a particular order, embodiments contemplate actions performed in any suitable order or combination according to particular needs.

At action 1302, in an embodiment, 3D mapping system 140 acquires a real-time tissue image. Imaging module 204 controls imaging system 130 to generate a real-time tissue image, and stores the real-time tissue image in images data 232. Graphical user interface module 206 accesses images data 232 and displays the real-time tissue image on display device 148.

At action 1304, in an embodiment, treatment module 220 accesses the treatment plan stored in treatment data 244, the 3D tissue model stored in 3D model data 234, and alignment coordinates stored in alignment data 236. Treatment module 220 uses the alignment coordinates to superimpose the 3D tissue model, containing 3D lesion cylinders 1244 representing the one or more tissue regions to be treated according to the treatment plan, over the real-time tissue image on display device 148. In this manner, treatment module 220 permits the visualization of the tissue regions that will be treated according to the treatment plan against the real-time image of the tissue of patient 160.

At action 1306, according to embodiments, a medical professional uses the real-time tissue image, 3D tissue model, and treatment plan displayed on display device 148 to treat one or more tissue regions according to the treatment plan. In an embodiment, treatment module 220 may update treatment plan data 244 in response to the actions of a medical professional, such as, by example, by storing in treatment plan data 244 and alignment data 236 the alignment coordinates of tissue treated by the medical professional. Following treatment, treatment module 220 terminates method 1300.

Reference in the foregoing specification to “one embodiment”, “an embodiment”, or “some embodiments” means that a particular feature, structure, or characteristic described in connection with the embodiment is included in at least one embodiment of the invention. The appearances of the phrase “in one embodiment” in various places in the specification are not necessarily all referring to the same embodiment.

While the exemplary embodiments have been shown and described, it will be understood that various changes and modifications to the foregoing embodiments may become apparent to those skilled in the art without departing from the spirit and scope of the present invention.

1. A non-transitory computer-readable storage medium stored therein computer-readable instructions, the computer-readable instructions when executed configured to:
access one or more tissue images comprising animal tissue;
generate a three-dimensional tissue model from the one or more tissue images;
develop a biopsy plan to collect tissue specimens from one or more tissue specimen collection sites corresponding to the three-dimensional tissue model, the computer-readable instructions when executed configuring a selectable length of tissue specimen to be collected at each tissue specimen collection site;
determine whether one or more regions of tissue on the three-dimensional tissue model are not sampled by a tissue specimen collection site;
display, on the three-dimensional tissue model, biopsy results associated with each of the one or more collected tissue specimens, the biopsy results displaying, if one or more tissue lesions are present within a collected tissue specimen, the location of each of the one or more tissue lesions along the length of the collected tissue specimen; and
generate a treatment plan to treat one or more tissue regions of animal in response to the biopsy results.

2. The non-transitory computer-readable storage medium of claim 1, wherein the computer-readable instructions when executed are further configured to, after developing the biopsy plan to collect tissue specimens from the one or more tissue specimen collection sites on the three-dimensional tissue model:
modify the biopsy plan to alter one or more of the number or location of the one or more tissue specimen collection sites, volume of tissue collected from each tissue specimen collection site, or length of tissue specimen collected from each tissue specimen collection site.

3. The non-transitory computer-readable storage medium of claim 1, wherein the computer-readable instructions when executed are further configured to, after developing the biopsy plan to collect tissue specimens from the one or more tissue specimen collection sites on the three-dimensional tissue model, and before displaying on the three-dimensional tissue model the biopsy results associated with each of the one or more collected tissue specimens:
align location of the one or more tissue specimen collection sites on the three-dimensional tissue model with one or more tissue images.

4. The non-transitory computer-readable storage medium of claim 1, wherein the computer-readable instructions when executed are further configured to, after displaying biopsy results associated with one or more tissue specimens collected from each of the one or more tissue specimen collection sites on the three-dimensional tissue model:
display on the three-dimensional tissue model one or more of size, location, or severity of one or more lesions detected in the biopsy results.

5. The non-transitory computer-readable storage medium of claim 4, wherein the computer-readable instructions when executed are further configured to, after displaying on the three-dimensional tissue model the one or more of the size, location, or severity of the one or more lesions detected in the biopsy results:
generate and display a radius of treatment sufficient to encompass for treatment the one or more lesions detected in the biopsy results.

6. The non-transitory computer-readable storage medium of claim 5, wherein the computer-readable instructions when executed are further configured to, after generating and displaying the radius of treatment sufficient to encompass for treatment the one or more lesions detected in the biopsy results:
expand the radius of treatment to encompass one or more lesion-free tissue specimen collection sites.

7. A system comprising:
a needle assembly;
an actuator assembly connected to the needle assembly;
an imaging system; and
a computer coupled with a database and comprising a processor, memory, and a display device, the computer configured to generate and implement a biopsy plan by:
operating the imaging system to acquire one or more tissue images from an animal;
generating a three-dimensional tissue model from the one or more tissue images;
developing a biopsy plan to collect tissue specimens from one or more tissue specimen collection sites on the three-dimensional tissue model, the computer configuring a selectable length of tissue specimen to be collected at each tissue specimen collection site;
determining whether one or more regions of tissue on the three-dimensional tissue model are not sampled by a tissue specimen collection site;
actuating the actuator assembly to extend the needle assembly into the animal at locations on the animal that correspond to the tissue specimen collection sites specified by the biopsy plan, collect a tissue specimen of selected length with the needle assembly, and retract the needle assembly;
displaying on the three-dimensional tissue model, using the display device, biopsy results associated with each of the one or more collected tissue specimens, the biopsy results displaying, if one or more tissue lesions are present within a collected tissue specimen, the location of each of the one or more tissue lesions along the length of the collected tissue specimen; and
generating a treatment plan to treat one or more tissue regions of animal.

8. The system of claim 7, further comprising, after the computer develops the biopsy plan to collect tissue specimens from the one or more tissue specimen collection sites on the three-dimensional tissue model:
modifying the biopsy plan to alter one or more of diameter of the needle assembly, number or location of tissue specimen collection sites from which the needle assembly will collect tissue specimens, or length of tissue specimen collected from each tissue specimen collection site.

9. The system of claim 7, further comprising, after the computer actuates the actuator assembly to extend the needle assembly into the animal:
verifying, using the imaging system, that location of the needle assembly in animal corresponds to a tissue specimen collection site specified by the biopsy plan.

10. The system of claim 9, further comprising, after verifying that location of the needle assembly in animal corresponds to a tissue specimen collection site specified by the biopsy plan:
modifying length of tissue specimen collected from location of the needle assembly in response to the verified location of the needle assembly.

11. The system of claim 9, further comprising, after verifying that location of the needle assembly in animal corresponds to a tissue specimen collection site specified by the biopsy plan:
modifying number or location of tissue specimen collection sites from which the needle assembly will collect tissue specimens.

12. The system of claim 7, further comprising, after displaying on the three-dimensional tissue model, using the display device, the biopsy results associated with each of the one or more collected tissue specimens:
displaying on the three-dimensional tissue model one or more of size, location, or severity of one or more lesions detected in the biopsy results.

13. The system of claim 12, wherein the treatment plan specifies treatment of all tissue regions in which one or more lesions were detected in the biopsy results and all tissue regions adjacent to all tissue regions in which one or more lesions were detected in the biopsy results.

14. A method comprising:
operating, using a computer comprising a processor, memory, and a display device and coupled to an imaging system, the imaging system to acquire one or more tissue images from an animal;
generating, with the computer processor, a three-dimensional tissue model from the one or more tissue images;
developing, with the computer processor, a biopsy plan to collect tissue specimens from one or more tissue specimen collection sites on the three-dimensional tissue model, the computer processor configuring a selectable length of tissue specimen to be collected at each tissue specimen collection site;
determining, with the computer processor, whether one or more regions of tissue on the three-dimensional tissue model are not sampled by a tissue specimen collection site;
the computer processor actuating an actuator assembly connected to a needle assembly to:
extend the needle assembly into the animal at locations on the animal that correspond to the tissue specimen collection sites specified by the biopsy plan;
collect a tissue specimen of selected length with the needle assembly; and
retract the needle assembly;
displaying on the three-dimensional tissue model, using the display device, biopsy results associated with each of the one or more collected tissue specimens, the biopsy results displaying, if one or more tissue lesions are present within a collected tissue specimen, the location of each of the one or more tissue lesions along the length of the collected tissue specimen; and
generating, with the computer processor, a treatment plan to treat one or more tissue regions of animal.

15. The method of claim 14, further comprising, after developing, with the computer processor, a biopsy plan to collect tissue specimens from one or more tissue specimen collection sites on the three-dimensional tissue model:
modifying, with the computer processor, the biopsy plan to alter one or more of diameter of the needle assembly, number or location of tissue specimen collection sites from which the needle assembly will collect tissue specimens, or length of tissue specimen collected from each tissue specimen collection site.

16. The method of claim 14, further comprising:
verifying, using the imaging system, that location of the needle assembly in animal corresponds to a tissue specimen collection site specified by the biopsy plan.

17. The method of claim 16, further comprising, after verifying that location of the needle assembly in animal corresponds to a tissue specimen collection site specified by the biopsy plan:
modifying length of tissue specimen collected from location of the needle assembly in response to the verified location of the needle assembly.

18. The method of claim 16, further comprising, after verifying that location of the needle assembly in animal corresponds to a tissue specimen collection site specified by the biopsy plan:
modifying number or location of tissue specimen collection sites from which the needle assembly will collect tissue specimens.

19. The method of claim 14, further comprising, after displaying on the three-dimensional tissue model, using the display device, the biopsy results associated with each of the one or more collected tissue:
displaying on the three-dimensional tissue model one or more of size, location, or severity of one or more lesions detected in the biopsy results.

20. The method of claim 19, wherein the treatment plan specifies treatment of all tissue regions in which one or more lesions were detected in the biopsy results and all tissue regions adjacent to all tissue regions in which one or more lesions were detected in the biopsy results.
